(19) United States
(12) Patent Application Publication
(10) Pub. No.: US 2025/0064808 A1
(43) Pub. Date: Feb. 27, 2025

(54) METHODS FOR THE TREATMENT OF NLRP3 RELATED DISORDERS

(71) Applicant: The Regents of the University of California, Oakland, CA (US)

(72) Inventors: Michael Karin, La Jolla, CA (US); Hongxu Xian, La Jolla, CA (US)

(21) Appl. No.: 18/942,238

(22) Filed: Nov. 8, 2024

Related U.S. Application Data
(63) Continuation of application No. PCT/US23/21375, filed on May 8, 2023.
(60) Provisional application No. 63/339,762, filed on May 9, 2022.

(51) Int. Cl. A61K 31/517; A61P 37/06; C12N 15/113

(52) U.S. Cl. CPC A61K 31/517; A61P 37/06; C12N 15/1137; C12N 2310/14; C12N 2310/531

(57) ABSTRACT

Provided herein are methods for treating a disease or condition associated with one or more of: mtDNA fragmentation; cytostolic Ox-mtDNA; mtDNA release; or Casp1 activation and IL-1β production by administering an effective amount of an inhibitor of human flap endonuclease-1 (FEN1). These diseases include without limitation, an inflammatory or a neurodegenerative disease, such as Alzheimer's, disease, Parkinson's disease, atherosclerosis, NASH, asthma, inflammatory bowel disease, melanoma, metabolic pathologies, including obesity, type 2 diabetes, atherosclerosis, inflammatory bowel diseases, gout, and periodic fevers.

### CROSS-REFERENCE TO RELATED APPLICATION

This application is a U.S. Continuation of PCT International Application No. PCT/US2023/021375, filed on May 8, 2023, which claims priority under 35 U.S.C. § 119(e) to U.S. Provisional Application No. 63/339,762, filed May 9, 2022, the contents of which are incorporated herein by reference in their entireties.

### STATEMENT OF GOVERNMENT SUPPORT

This invention was made with government support under AI043477 awarded by the National Institutes of Health (NIH). The government has certain rights in the invention.

### SEQUENCE LISTING

The instant application contains a Sequence Listing which has been submitted electronically in XML file format and is hereby incorporated by reference in its entirety. Said XML copy, created on Nov. 5, 2024, is named 114198-0777_SL.xml and is 37,232 bytes in size.

### BACKGROUND OF THE DISCLOSURE

Inflammation encompasses a series of protective reactions mounted by the evolutionarily conserved innate immune system that benefit the host by eliminating pathogens and restoring cellular and organismal homeostasis (Karin and Clevers, 2016; Kotas and Medzhitov, 2015). However, when not properly terminated inflammation ceases to be beneficial and becomes pathogenic. In response to chemically and physically diverse triggers of cell injury, the NOD-, LRR- and pyrin domain-containing protein 3 (NLRP3) inflammasome (NLRP3Inf), a key sensor and effector of tissue damage, is activated resulting in caspase-1 (Casp1)-mediated IL-1β and IL-18 secretion (Gross et al., 2011). The NLRP3Inf is a large cytosolic complex formed by the sensor NLRP3, the adaptor apoptosis-associated speck-like protein containing a CARD (ASC), the protein kinase NEK7 and the effector pro-Casp1. After sterile injury, NLRP3 exposes its pyrin domain (PYD) to bind ASC, which uses its CARD domain to recruit pro-Casp1, triggering its self-cleavage and releasing its active p20 fragment that converts pro-IL-1β and pro-IL-18 to their mature and proinflammatory forms (Gross et al., 2011; Lu et al., 2014). Recent studies have shed further light on this process, revealing that inactive NLRP3 forms a double ring cage held together by LRR-LRR interactions that shield the PYD and prevent premature activation (Andreeva et al., 2021). Yet, the precise pathway leading to NLRP3Inf assembly and activation after cell injury remains nebulous. Importantly, persistent NLRP3 signaling underlies many chronic diseases and metabolic pathologies, including obesity, type 2 diabetes, atherosclerosis, inflammatory bowel diseases, gout, and periodic fevers, as well as neurodegenerative disorders, including Parkinson's and Alzheimer's diseases (AD) (Halle et al., 2008; Heneka et al., 2015; Lamkanfi and Dixit, 2012; Mangan et al., 2018). Thus, a need exists in the art for safe and effective treatments for these and associated diseases.

### SUMMARY OF THE DISCLOSURE

In one aspect, this disclosure provides a method for one or more in a mammalian cell: inhibition of mtDNA fragmentation; inhibition of cytostolic Ox-mtDNA; inhibition of mtDNA release; or inhibition of Casp1 activation and IL-1β production, comprising contacting the mammalian cell with an effective amount of an inhibitor of human flap endonuclease-1 (FEN1), thereby inhibiting mtDNA fragmentation; inhibiting cytostolic Ox-mtDNA; inhibiting mtDNA release; or inhibiting of Casp1 activation and IL-1β production.

Also provided is a method for treating a disease or condition associated with mtDNA fragmentation; cytostolic Ox-mtDNA; mtDNA release; or Casp1 activation and IL-1β production in a subject in need thereof, comprising administering to the subject an effective amount of an inhibitor of human flap endonuclease-1 (FEN1), thereby treating the disease or condition associated with mtDNA fragmentation; cytostolic Ox-mtDNA; mtDNA release; or Casp1 activation and IL-1β production in the subject. Non-limiting examples of such disease or condition is selected from chronic diseases and metabolic pathologies, optionally obesity, type 2 diabetes, atherosclerosis, inflammatory bowel diseases, gout, and periodic fevers, as well as neurodegenerative disorders, including Parkinson's and Alzheimer's diseases (AD), atherosclerosis, NASH, asthma, inflammatory bowel disease, or melanoma.

### BRIEF DESCRIPTION OF THE DRAWING

FIGS. 1A-1M: Base excision repair inhibits Ox-mtDNA production and attenuates NLRP3 inflammasome activation (FIG. 1A) H&E, Sirius red, F4/80 and myeloperoxidase (MPO) antibody staining of lung tissue from mice challenged with 5 mg/kg LPS 24 h prior to tissue collection. n=3 for mock treatment and n=6-7 mice for LPS treatment. 10-12 images per mouse were analyzed. Scale bar, 100 μm. (FIG. 1B) Area (in %) occupied by F4/80 or MPO positive cells in lung sections from (FIG. 1A). Data are means±s.d. (FIG. 1C) IL-1β and TNF concentrations in BALF from (FIG. 1A) measured by ELISA. Data are means±s.d. (FIG. 1D and FIG. 1E) 8-OH-dG content of mtDNA from cytosol (left) or mitochondria (right) of LPS (200 ng/mL, 4 h)-primed WT and mt-OggTg (FIG. 1D) or WT and Ogg1−/− (FIG. 1E) bone marrow derived macrophages (BMDM) stimulated−/+ATP (4 mM, 1 h). (FIG. 1F) Immunoblot (IB) analysis of lysates of WT and Ogg1−/− BMDM. (FIG. 1G and FIG. 1H) Relative cytosolic mtDNA amounts in LPS-primed WT and mt-OggTg (FIG. 1G) or WT and Ogg1−/− (FIG. 1H) BMDM stimulated−/+ATP. The relative ratios of D-loop mtDNA, Cox1 mtDNA, or non-NUMT mtDNA are shown. (FIG. 1I) IB analysis of Casp1 p20 and mature IL-1β in supernatants and NLRP3, Pro-IL-1β, Pro-Casp1, and ASC in lysates of LPS-primed WT or mt-Ogg1Tg BMDM stimulated−/+ATP (4 mM, 1 h) or nigericin (Nig) (10 μM, 1h) (left). Mitochondrial OGG1 IB in WT and mt-Ogg1Tg BMDM (right) is shown. (FIG. 1J) IL-1β and TNF secretion by LPS-primed WT or mt-Ogg1Tg BMDM challenged with different NLRP3 activators (ATP, 4 mM for 1 h, nigericin, 10 μM for 1 h, MSU, 600 μg/mL for 6 h and alum, 500 μg/mL for 6 h). (FIG. 1K) IB of Casp1 p20 and mature IL-1β in supernatants of LPS-primed WT and Ogg1−/− BMDM stimulated−/+ATP (4 mM) or nigericin (10 μM) for 1 h. (FIG. 1L) Representative fluorescent microscopy images of WT or mt-Ogg1Tg BMDM co-stained for Atp5b and ASC before or after LPS priming followed by ATP (4 mM) or nigericin (10 μM) stimulation for 1 h. DAPI stains nuclei. Arrows indicate ASC specks. Scale bar, 5 μm. (FIG. 1M) Percentages of cells shown in (FIG. 1L) with ASC specks. n=150 cells per group from 3 independent experiments. IBs are one representative out of 3 independent experiments. Results in (FIG. 1D, FIG. 1E, FIG. 1G, FIG. 1H, FIG. 1J and FIG. 1M) are mean±s.d. (n=3). *p<0.05; **p<0.01; ***p<0.001. ns, not significant. Two-sided unpaired t-test. See also FIG. 8.

FIGS. 2A-2M: mPTP opening and VDAC oligomerization enable mtDNA cytosolic release and NLRP3 inflammasome activation (FIG. 2A) VDAC oligomerization in BMDM that were pretreated−/+VBIT-4 (10 μM, 16 h), primed−/+LPS (200 ng/ml, 4 h) and stimulated−/+ATP (4 mM, 1 h) was determined by IB analysis of cell lysates incubated with the cross-linking agent EGS (200 μM, 40 min, 30° C.) to stabilize VDAC oligomers. Asterisk indicates nonspecific band. (FIG. 2B) Relative calcein fluorescence in BMDM pretreated−/+CsA (1 μM, 16 h) or VBIT-4 (10 μM, 16 h), followed by priming−/+LPS (200 ng/mL, 4 h) and stimulation−/+ATP (4 mM, 1 h). The calcein-quenching assay measures mPTP opening. (FIG. 2C and FIG. 2D) Relative amounts of Ox-mtDNA (FIG. 2C) or total mtDNA (FIG. 2D) in cytosols of LPS-primed shCtrl- or shOgg1-transduced BMDM, pretreated−/+CsA and VBIT-4 and stimulated with ATP as in (FIG. 2B). (FIG. 2E) VDAC oligomerization in BMDM treated−/+EtBr (450 ng/mL, 4 days) to deplete mtDNA, and subsequently incubated−/+ATP (4 mM) for 1, 2 or 3 h was analyzed as in (FIG. 2A). Asterisk indicates nonspecific band. (FIG. 2F) Experimental scheme for measuring Ox-mtDNA in the intermembrane space (IMS) based on OMM rupture with proteinase K. (FIGS. G and H) Relative amounts of Ox-mtDNA (FIG. 2G) or total mtDNA (FIG. 2H) in supernatants (IMS fraction) of proteinase K digested mitochondria isolated from BMDM treated as indicated. (FIG. 2I) IB analysis of Casp1 p20 and mature IL-1β in supernatants of LPS-primed BMDM, pretreated−/+CsA (1 μM, 16 h) and VBIT-4 (10 μM, 16 h) and challenged with ATP (4 mM, 1 h). (FIG. 2J) Relative Casp1 p20 and IL-1β in supernatants of BMDM treated as in (FIG. 2I). (FIG. 2K) IL-1β and TNF secretion by BMDM primed−/+LPS (200 ng/ml, 4 h), pretreated−/+CsA (1 μM, 16 h) and VBIT-4 (10 μM, 16 h) and challenged with different NLRP3 activators. (FIG. 2L) Representative fluorescent microscopy images of BMDM, pretreated−/+VBIT-4 (10 μM, 16 h), primed−/+LPS (200 ng/ml, 4 h), stimulated−/+ATP (4 mM, 1 h) or nigericin (10 μM, 1 h) and co-stained for Atp5b and ASC. Arrows indicate ASC specks. Scale bar, 5 μm. (FIG. 2M) Percentages of cells from (FIG. 2L) with ASC specks. n=150 cells per group from 3 independent experiments. IBs show one representative out of 3 independent experiments. Results in (FIGS. 2B-2D, FIG. 2G, FIG. 2H, FIG. 2J, FIG. 2K, and FIG. 2M) are mean±s.d. (n=3). *p<0.05; **p<0.01; ***p<0.001. ns, not significant. Two-sided unpaired t-test. See also FIG. 9.

FIGS. 3A-3F: [Ca²⁺]m uptake initiates mPTP opening and VDAC oligomerization to release Ox-mtDNA (FIG. 3A) VDAC oligomerization in BMDM stimulated with different NLRP3 activators was analyzed as in FIG. 2A. Asterisk indicates nonspecific band. (FIG. 3B) Relative VDAC oligomerization in cells from (FIG. 3A) (n=3). (FIG. 3C) Relative calcein fluorescence in BMDM−/+LPS (200 ng/mL, 4 h) priming and challenged with different NLRP3 agonists, using the calcein-quenching assay to measure mPTP opening as in FIG. 2B (n=4). (FIG. 3D) Representative fluorescent microscopy images of BMDM double-labeled with MitoTracker Green and Rhod-2 to detect [Ca²⁺]m, followed by ATP (2^{nd} line from top), nigericin (top line), MSU (third line from top) or alum stimulation (bottom line). Scale bar, 10 μm. (FIG. 3E) Normalized Rhod-2 fluorescence traces of BMDM challenged with ATP, nigericin, MSU or alum at 40 seconds (n=8). (FIG. 3F) VDAC oligomerization in BMDM pretreated−/+CsA (1 μM, 16 h), followed by priming−/+LPS and stimulation−/+ATP was analyzed as in FIG. 2A. IBs are one representative out of 3. Results in (FIG. 3B, FIG. 3C, and FIG. 3E) are mean±s.d. *p<0.05; **p<0.01; ***p<0.001. Two-sided unpaired t-test. See also FIG. 10.

FIGS. 4A-4H: MCU mediates mitochondrial Ca²⁺ uptake to trigger mPTP-VDAC channel opening and Ox-mtDNA cytosolic release (FIG. 4A) Relative Rhod-2 fluorescence in LPS-primed BMDM pretreated−/+the IP3R inhibitor xestospongin C (XeC) (5 μM, 16 h) or the MCU inhibitor ruthenium red (RuR) (10 μM, 16 h), followed by ATP (4 mM, 1 h) or nigericin (10 μM, 1 h) challenge (n=3). (FIG. 4B) Relative calcein fluorescence in BMDM treated as in (FIG. 4A) (n=3). (FIG. 4C) VDAC oligomerization in BMDM treated as in (FIG. 4A). (FIG. 4D and FIG. 4E) Amounts of Ox-mtDNA (FIG. 4D) or relative total mtDNA (FIG. 4E) in cytosols of LPS-primed BMDM pretreated−/+XeC (5 μM, 16 h) or RuR (10 μM, 16 h) and stimulated with ATP (4 mM, 1 h) (n=3). (FIG. 4F) IB analysis of Casp1 p20 and IL-Iβ in supernatants of BMDM−/+RuR pretreatment, primed−/+LPS, and stimulated with ATP or nigericin as in (FIG. 4A). (FIG. 4G) IL-1B and TNF secretion by BMDM pretreated−/+RuR (10 μM, 16 h), primed or not with LPS (200 ng/ml, 4 h) and challenged with different NLRP3 activators (n=3). (FIG. 4H) IB analysis of NLRP3, pro-IL-1B, pro-Casp1, ASC, and tubulin in lysates of BMDM pretreated−/+RuR (10 μM, 16 h), before and after LPS priming (200 ng/ml, 4 h). IBs are one representative out of 3. Results in (FIG. 4A, FIG. 4B, FIG. 4D, FIG. 4E, and FIG. 4G) are mean±s.d. *p<0.05; **p<0.01; ***p<0.001. ns, not significant. Two-sided unpaired t-test. See also FIG. 11.

FIGS. 5A-5O: FEN1-mediated mtDNA fragmentation is required for Ox-mtDNA cytosolic release and NLRP3 inflammasome activation (FIG. 5A) Agarose gel electrophoresis of cytosolic (Cyto) and mitochondrial (Mito) DNA from BMDM−/+LPS priming (200 ng/ml), −/+nigericin (Nig) (10 μM) or ATP (4 mM) stimulation for 1 h. (FIG. 5B) Agarose gel electrophoresis of cytosolic and mitochondrial mtDNA PCR amplification products (591 bp and 5698 bp) form BMDM−/+LPS priming that were challenged with different NLRP3 activators (n=3). (FIG. 5C) IB analysis of Casp1 p20 and IL-1β in culture supernatants of LPS (200 ng/ml, 4 h)-primed BMDM that were transduced with shCtrl, shMgme1, shMre11a, or shFen1 and stimulated−/+ATP (4 mM, 1 h). (FIG. 5D) IB analysis of MGME1, MRE11A, FEN1 and Tom20 in mitochondria isolated from BMDM transduced with shCtrl, shMgme1, shMre11a, or shFen1. (FIG. 5E and (FIG. 5F) Cytosolic Ox-mtDNA (n=10) (FIG. 5E) or relative mtDNA amount (n=3) (FIG. 5F) in BMDM that were transduced with shCtrl, shMgme1 and shFen1 treated as in (FIG. 5C). (FIG. 5G) Analysis of Cyto and Mito DNA from LPS-primed shCtrl- or shFen1-transduced BMDM stimulated−/+ATP as in (FIG. 5C). (H) mtDNA PCR amplification products (entire genome, 16299 bp, or D-loop region, 591 bp) from LPS-primed BMDM stimulated−/+ATP or nigericin as in (FIG. 5A). (FIG. 5I) Relative amounts of whole mtDNA genome in the BMDM analyzed in (FIG. 5H). (FIG. 5J) Analysis of Cyto or Mito DNA from BMDM that were pretreated with FEN1-IN-4 (10 μM, 16 h), LPS-primed and stimulated−/+ATP. (FIG. 5K and FIG. 5L) Amounts of Ox-mtDNA (FIG. 5K) or relative total mtDNA (FIG. 5L) in the cytosol of BMDM treated as in (FIG. 5J) (n=3). (FIG. 5M) IB analysis of Casp1 p20 and mature IL-1β in culture supernatants of BMDM pretreated−/+FEN1-IN-4, primed with LPS and challenged with ATP or nigericin. (FIG. 5N) Peritoneal IL-1β and TNF in mice treated with FEN1-IN-4 (40 mg/kg) 16 h prior to i.p. injections of alum (1 mg) or PBS, whose peritoneal exudates were analyzed 4 h later. Data are mean±s.d. (n=6 PBS- and alum-injected groups; n=8 in alum+FEN1-IN-4-treated group). (FIG. 5O) Alum-induced peritoneal exudate cells (PEC), neutrophils (CD11b+Ly6G+F4/80−) and monocytes (CD11b+Ly6C+Ly6G−) in mice pretreated with FEN1-IN-4 at 40 mg/kg 16 h prior to i.p. injections of alum or PBS for 16 h. (n=6 PBS- and alum-treated groups; n=8 alum+FEN1-IN-4-treated group). The EtBr-stained agarose gels in (FIG. 5A, FIG. 5B, FIG. 5G, FIG. 5H, and FIG. 5J) are representative of 3 independent experiments. IBs are one representative out of 3. Results in (FIG. 5E, FIG. 5F, FIG. 5I, FIG. 5K, FIG. 5L, FIG. 5N and FIG. 5O) are mean±s.d. *p<0.05; **p<0.01; ***p<0.001. ns, not significant. Two-sided unpaired t-test. See also FIG. 12.

FIGS. 6A-6J: mt-OGG1 and FEN1-IN-4 restrain cell-free Ox-mtDNA secretion (FIG. 6A) Relative ccf-mtDNA amounts in BALF from FIG. 1A (n=3 for PBS-treated groups; n=6-7 for LPS-treated groups). (FIG. 6B) Relative ccf-mtDNA amounts in peritoneal exudate from FIG. 8A (n=4 for PBS-treated groups; n=6 for alum-treated groups). (FIG. 6C) Relative ratio of Fpg-treated (+) to nontreated (−) ccf-mtDNA from (FIG. 6B), indicating the fraction of cell-free non-Ox-mtDNA (Fpg-resistant) relative to total secreted mtDNA. (FIG. 6D and FIG. 6E) Relative amounts of ccf-mtDNA (FIG. 6D) and cell-free non-Ox-mtDNA (Fpg-resistant) (FIG. 6E) in peritoneal exudates treated as in FIG. 5N. (n=4-5 for PBS- and alum-treated groups; n=9 for alum+FEN1-IN-4-treated group). (FIG. 6F) Experimental scheme for measuring the signaling activity of secreted ccf-mtDNA. (FIG. 6G and FIG. 6H) Relative amounts of secreted mtDNA (FIG. 6G) or Ox-mtDNA (FIG. 6H) from LPS-primed BMDM challenged with different NLRP3 activators (n=3). (FIG. 6I) Q-PCR quantitation of Il1b, I16, Tnf, Cxcl10, Isg15, Ifit3 and Irf7 mRNAs in recipient BMDM incubated for 24 h with DNA (−/+DNase I digestion) secreted by LPS-primed donor BMDM challenged with ATP or nigericin (n=3). (FIG. 6J) Q-PCR quantitation of Il1b, Il6, Isg15, Ifit3 and Irf7 mRNAs in recipient BMDM incubated for 24 h with DNA secreted by donor BMDM treated as in Figure S6H (n=3). Results are mean±s.d. *p<0.05; **p<0.01; ***p<0.001. Two-sided unpaired t-test. See also FIG. 13.

FIGS. 7A-7F: Induction of mtDNA release by NLRP3 Inflammasome activators leads to STING Ser³⁶⁵ phosphorylation (FIG. 7A) Representative fluorescent microscopy images of LPS (200 ng/mL, 4 h)-primed WT or mt-Ogg1Tg BMDM stained for phospho-STING (Ser³⁶⁵) before or after ATP (4 mM) or nigericin (10 μM) stimulation for 1 h. DAPI stains nuclei. Scale bar, 5 μm. (FIG. 7B) Percentages of cells from (FIG. 7A) with p-STING (Ser³⁶⁵) puncta. n=150 cells per group from 3 independent experiments. (FIG. 7C) IB analysis of STING Ser³⁶⁵ phosphorylation in lysates and Casp1 p20 and mature IL-Iβ in supernatants of BMDM pretreated−/+EtBr (450 ng/ml, 4 days) to deplete mtDNA, followed by LPS priming and−/+ATP stimulation as in (FIG. 7A). (FIG. 7D) Relative p-STING (Ser³⁶⁵) to total STING amounts from cells in (FIG. 7C). (E) IB analysis of STING Ser³⁶⁵ phosphorylation and NLRP3 in lysates and Casp1 p20 and mature IL-1β in supernatants of LPS-primed WT and Nlrp3−/−BMDM that were pretreated−/+CsA (1 μM, 16 hrs), VBIT-4 (10 μM, 16 hrs) or FEN1-IN-4 (10 μM, 16 hrs) and stimulated−/+ATP as in (FIG. 7A). (FIG. 7F) Relative p-STING (Ser³⁶⁵) to total STING amounts from cells in (FIG. 7E). IBs are one representative out of 3. Results in (FIG. 7B, FIG. 7B, and FIG. 7F) are mean±s.d. *p<0.05; **p<0.01; ***p<0.001. Two-sided unpaired t-test. See also FIG. 14.

FIGS. 8A-8T: mt-OGG1 reduces Ox-mtDNA content and restrains NLRP3Inf activation, related to FIG. 1 (FIG. 8A) Peritoneal IL-1 and TNF in WT or mt-OggTg mice 4 hrs after i.p. injection of alum (1 mg) or PBS. Data are mean±s.d. (n=5 in PBS-treated group; n=6 in alum-treated group). (FIG. 8B) Alum-induced infiltration of peritoneal exudate cells (PEC), neutrophils (CD11b+Ly6G+F4/80−) and monocytes (CD11b+Ly6C+Ly6G−) in WT and mt-OggTg mice 16 hrs after alum (300 ug) or PBS injection. Data are mean±s.d. (n=5 for each group). (FIG. 8C) 8-OH-dG amounts in mtDNA from cytosol (left) and mitochondria (right) of LPSprimed BMDM transduced with Ogg1 (shOgg1 #1 and shOgg1 #2) or control (shCtrl) shRNA, before and after ATP stimulation. (FIG. 8D) IB analysis of OGG1 and Tom20 in mitochondria isolated from shCtrl- or shOgg1 RNA-transduced BMDM. (FIG. 8E) Relative cytosolic mtDNA amounts in BMDM treated as in (FIG. 8C). (F-H) Relative total mtDNA amounts in WT and mt-OggTg (FIG. 8F), WT and Ogg1−/− (FIG. 8G) or shCtrl- and shOgg1 RNA-transduced BMDM (FIG. 8H), before and after LPS priming. The ratios of D-loop mtDNA to Tert nDNA, Cox1 mtDNA to 18S nDNA, or mtDNA that is not inserted into nuclear DNA (non-NUMT) to B2m nDNA are shown. (FIG. 8I) Relative Casp1 p20 and IL-1β in culture supernatants of WT and mt-OggTg BMDM treated as in FIG. 1I. (FIG. 8J) IB analysis of Casp1 p20 and IL-1β in culture supernatants of shCtrl- or shOgg1 RNA40 transduced BMDM treated as indicated. (FIG. 8K) Relative Casp1 p20 and IL-1β in supernatants of BMDM shown in (FIG. 8J). (FIG. 8L) IB analysis of NLRP3, pro-IL-1β, pro-caspase-1, ASC and Ogg1 in shCtrl- and shOgg1 RNA-transduced BMDM, before and after LPS priming. Tubulin is loading control. (FIG. 8M) IB analysis of cleaved caspase 9 (Casp9) and cleaved caspase 3 (Casp3) in lysates of WT and mt-OggTg BMDM incubated−/+staurosporine (STS, 100 nM), etoposide (10 μM) or camptothecin (10 μM) for 16 hrs (left). Mitochondrial OGG1 in WT and mt-OggTg BMDM (right). (FIG. 8N) Relative amounts of cleaved Casp9 and Casp3 of BMDM shown in (FIG. 8M). (FIG. 8O) IB analysis of cleaved Casp9 and Casp3 in lysates of shCtrl- and shOgg1 RNA transduced BMDM treated as in (FIG. 8M). (FIG. 8P) Relative cleaved Casp9 and Casp3 in BMDM shown in (FIG. 8O). (FIG. 8Q) Representative fluorescent microscopy of shCtrl- and shOgg1 RNA-transduced BMDM treated as in (FIG. 8M) stained for Cyto C. Arrows indicate Cyto C release. Scale bar, 5 μm. (FIG. 8R) Percentages of cells from (FIG. 8Q) with Cyto C release. n=150 cells per group from 3 independent experiments. (FIG. 8S) IB analysis of cleaved Casp9 and Casp3 in lysates of shCtrl- and shOgg1 RNA-transduced BMDM treated as in (FIG. 8O). (FIG. 8T) Relative amounts of cleaved Casp9 and Casp3 in BMDM shown in (FIG. 8S). All IBs show one representative out of 3. Results in (FIG. 8C, FIGS. 8E-8I, FIG. 8K, FIG. 8N, FIG. 8P, FIG. 8R, and FIG. 8T) are mean±s.d. (n=3). *p<0.05; **p<0.01; ***p<0.001. ns, not significant. Two-sided unpaired t-test.

FIGS. 9A-9L: CsA and VBIT-4 restrict NLRP3Inf activation, related to FIG. 2 (FIG. 9A) VDAC oligomerization in WT or mt-Ogg1^{Tg} BMDM that were primed−/+LPS and stimulated−/+ATP was analyzed as in FIG. 2A. Asterisk indicates nonspecific band. (FIG. 9B and FIG. 9C) Relative calcein fluorescent intensity in LPS-primed WT and mt-Ogg^{Tg} (FIG. 9B) or shCtrl- or shOgg1 RNA-transduced (FIG. 9C) BMDM stimulated−/+ATP or nigericin, using the calcein-quenching assay for mPTP opening (n=5). (FIG. 9D) Relative mtDNA amounts in BMDM from FIG. 2E, before or after LPS priming, demonstrating the efficacy of mtDNA depletion by EtBr. (FIG. 9E) Relative VDAC oligomerization in BMDM from FIG. 2E (n=3). (FIG. 9F) Mitochondria isolated from BMDM were digested with the indicated concentrations of proteinase K for 30 min on ice and IB analyzed with antibodies against the OMM proteins VDAC and Tom20 and the IMM proteins Tim23 and COX1/MT-CO1. (FIG. 9G) IB analysis of Casp1 p20 and IL-1β in supernatants of shCtrl- or shOgg1 RNAtransduced BMDM that were LPS primed and challenged with ATP, −/+CsA (1 μM, 16 hrs) or VBIT-4 (10 μM, 16 hrs) pretreatment. (FIG. 9H) IB analysis of NLRP3, pro-IL-1β, pro-caspase-1, ASC, and tubulin in lysates of BMDM before and after LPS priming, −/+CsA or VBIT-4 pretreatment as above. (FIG. 9I) IB analysis of Casp1 p20 and IL-1β in supernatants of LPS-primed BMDM transduced with shCtrl, shVdac1, shVdac3, or shVdac1+shVdac3 RNA, −/+ATP stimulation (top) and validation of VDAC silencing (bottom). (FIG. 9J) 8-OH-dG amounts in mtDNA from mitochondria of LPS-primed BMDM pretreated−/+CsA (1 μM, 16 hrs) or VBIT-4 (1 μM, 16 hrs), before and after ATP stimulation (n=6). (FIG. 9K) Relative mtROS amounts in LPS-primed BMDM pretreated−/+CsA or VBIT-4 as above and challenged with ATP or nigericin (n=6). (FIG. 9L) Mitochondrial membrane potential of BMDM treated as in (FIG. 9K) (n=3). All IBs show one representative out of 3. Results in (FIGS. 9B-9E ad FIGS. 9J-9L) are mean±s.d. *p<0.05; **p<0.01; ***p<0.001. ns, not significant. Two-sided unpaired t-test.

FIGS. 10A-10J: mtROS alone do not induce channel opening and consequent mtDNA release and apoptosis is not involved either, related to FIG. 3 (FIG. 10A) Relative Rhod-2 fluorescent intensity in BMDM that were challenged with different NLRP3 agonists, −/+LPS priming (n=3). (FIG. 10B and FIG. 10C) Relative Rhod-2 fluorescent intensity in WT and mt-Ogg^{Tg} (FIG. 10B) or shCtrl and shOgg1 transduced (FIG. 10C) BMDM before and after LPS priming, −/+ATP or nigericin stimulation (n=3). (FIG. 10D) Representative fluorescent microscopy images of LPS-primed BMDM−/+ATP or nigericin stimulation that were co-stained for cytochrome c (Cyto C) and ASC. DAPI stains nuclei. BMDM treated with etoposide were used as a positive control for Cyto C release. Arrows indicate ASC specks or Cyto C release. Scale bar, 5 μm. (E and F) Relative mtROS amounts (n=3) (FIG. 10E) and calcein fluorescent intensity (n=4) (FIG. 10F) in LPS-primed BMDM treated with ATP or the ROS generators DMNQ (20 μM, 16 hrs) and MitoParaquat (5 μM, 1 hr). (FIG. 10G) VDAC oligomerization in BMDM treated as in (FIG. 10E) was IB analyzed as in FIG. 9A. Asterisk indicates nonspecific band. (FIG. 10H) 8-OH-dG amounts in cytosolic (left) or mitochondrial (right) mtDNA in BMDM treated as in (FIG. 10E) (n=5). (FIG. 10I) Relative cytosolic mtDNA in BMDM treated as in (FIG. 10E) (n=3). (FIG. 10J) IB analysis of Casp1 p20 and IL-1β in supernatants and NLRP3, pro-IL-1β, ASC, procaspase-1, and tubulin in lysates of BMDM−/+LPS priming, followed by ATP, DMNQ or MitoParaquat treatment as above. All IBs show one representative out of 3. Results in (FIGS. 10A-10C, FIG. 10E, FIG. 10F, FIG. 10H and FIG. 10I) are mean±s.d. *p<0.05; **p<0.01; ***p<0.001. ns, not significant. Two-sided unpaired t-test.

FIGS. 11A-11D: MCU inhibitor blunts mPTP-VDAC channel opening and NLRP3Inf activation, related to FIG. 4 (FIG. 11A) Relative Fluo-4 fluorescent intensity of BMDM pretreated−/+XeC (5 μM, 16 hrs) and challenged with ATP (n=5). (FIG. 11B) Relative VDAC oligomerization in the BMDM from FIG. 4C (n=3). (FIG. 11C) Relative mtDNA amounts in BMDM pretreated−/+RuR (10 μM, 16 hrs) before and after LPS priming (n=3). (FIG. 11D) Relative Casp1 p20 and IL-1β in supernatants of BMDM from FIG. 4F (n=3). Results are mean±s.d. *p<0.05; **p<0.01. ns, not significant. Two-sided unpaired t-test.

FIGS. 12A-12N: Mgme1 is needed for mtDNA synthesis but FEN1 has no impact on total mtDNA content, Ox-mtDNA generation and pore opening, related to FIG. 5 (FIG. 12A) Relative mtDNA amounts in BMDM pretreated−/+EtBr (450 ng/ml, 4 days) before or after LPS priming. (FIG. 12B) Analysis of DNA in the cytosolic (Cyto), mitochondrial (Mito), or nuclear fractions of LPS-primed BMDM pretreated−/+EtBr (450 ng/ml, 4 days), followed by ATP or nigericin stimulation. The agarose gel is representative of 3 independent experiments. (FIG. 12C) PCR products generated by amplification of mtDNA (entire genome, 16299 bp or D-loop region, 591 bp) from BMDM before or after LPS priming, followed by stimulation by different NLRP3 activators. (FIG. 12D) Relative Casp1 p20 and IL-1β in supernatants of BMDM from FIG. 5C. (FIG. 12E) Relative mtDNA amounts in shCtrl, shMgme1, or shFen1 transduced BMDM−/+LPS priming. (FIG. 12F) Relative Casp1 p20 and IL-1β in supernatants of BMDM from FIG. 5M. (FIG. 12G) IL-1β and TNF secretion by BMDM pretreated−/+FEN1-IN-4 (10 μM, 16 hrs), primed−/+LPS, and stimulated with different NLRP3 activators (n=3). (FIG. 12H) IL-1β and TNF secretion by BMDM pretreated−/+FEN1-IN-4 (10 μM or 20 μM, 16 hrs), primed−/+LPS, followed by ATP challenge (n=3). (FIG. 12I) Relative mtDNA amounts in BMDM pretreated−/+FEN1-IN-4 as above, before or after LPS priming (n=3). (FIG. 12J and FIG. 12K) 8-OH-dG content of mtDNA from mitochondria (FIG. 12J) and relative mtROS amounts (FIG. 12K) in LPS-primed BMDM transduced with Fen1 (shFen1) or control (shCtrl) shRNAs (left) or pretreated−/+FEN1-IN-4 as above (right) and stimulated−/+ATP or nigericin (n=3-6). (FIG. 12L) Calcein fluorescent intensity of BMDM treated as in (FIG. 12JK (n=3). (FIG. 12M and FIG. 12N) VDAC oligomerization in LPS-primed BMDM transduced with shFen1 or shCtrl RNA (FIG. 12M) or pretreated−/+FEN1-IN-4 as above (FIG. 12N), and stimulated−/+ATP or nigericin, was IB analyzed as in FIG. 9A. Asterisk indicates nonspecific band. The agarose gel in (FIG. 12B and FIG. 12C) is representative of 3 independent experiments. Results in (FIG. 12A and FIGS. 12D-12L) are mean±s.d. *p<0.05; **p<0.01; ***p<0.001. ns, not significant. Two-sided unpaired t-test.

FIGS. 13A-13I: FEN1-IN-4 restrains cell-free Ox-mtDNA secretion via pyroptosis, related to FIG. 6 (FIG. 13A and FIG. 13B) Relative ccf-mtDNA amounts (FIG. 13A) and the ratio of Fpg-treated (+) to nontreated (−) mtDNA, indicating the fraction of cell-free non-Ox-mtDNA (Fpg-resistant) relative to total secreted mtDNA (FIG. 13B) in peritoneal exudates from FIG. 5O (n=4-5 for PBS- and alum-treated groups; n=6 for alum+FEN1-IN-4-treated group). (FIG. 13C) Relative amounts of extracellular Ox-mtDNA (left) or mtDNA in LPS-primed WT and Gsdmd−/−BMDM stimulated−/+ATP (n=3). (FIG. 13D) IB analysis of Casp1 p20, IL-1β and cleaved GSDMD [GSDMD NT (p30)] in culture supernatants of LPS-primed WT and Gsdmd−/−BMDM−/+ATP challenge (n=3) (FIG. 13E) 8-OH-dG amounts in mtDNA from mitochondria of LPS-primed WT or Gsdmd−/−BMDM before and after ATP stimulation (left). Relative total mtDNA amounts in WT and Gsdmd−/−BMDM, before and after LPS priming (n=3). (FIG. 13F) Relative amounts of intracellular (top panel) or secreted (bottom panel) mtDNA in BMDM pretreated−/+EtBr (450 ng/ml, 4 days), primed−/+LPS and stimulated−/+ATP or nigericin. Intracellular mtDNA content validates effective mtDNA depletion by EtBr (n=3). (FIG. 13G) Q-PCR quantitation of Il1b, Il6, Tnf, Cxcl10, Isg15, Ifit3 and Irf7 mRNAs in BMDM incubated for 24 hrs with cell free DNA collected from (FIG. 13F) (n=3). (FIG. 13H) Relative mtDNA amounts released by LPS-primed BMDM pretreated−/+CsA, VBIT-4 or FEN1-IN-4 as above and challenged with ATP or nigericin (n=3). (FIG. 13I) Q-PCR quantitation of Il1b, Il6, Tnf, Cxcl10, Isg15, Ifit3 and Irf7 mRNAs in BMDM−/+Pitstop 2 (2 uM) incubated for 24 hrs with extracellular DNA collected from LPS-primed donor BMDM stimulated−/+ATP or nigericin (n=4). Results are mean±s.d. *p<0.05; **p<0.01; ***p<0.001. ns, not significant. Two-sided unpaired t-test.

FIGS. 14A-14F: Induction of mtDNA release by NLRP3Inf activators leads to STING Ser³⁶⁵ phosphorylation, related to FIG. 7 (FIG. 14A) Q-PCR quantitation of Il1b, Il6, Tnf, Cxcl10, Isg15, Ifit3 and Irf7 mRNAs in WT, cGas−/− or Sting−/−BMDM incubated for 24 hrs with extracellular DNA collected from LPS-primed donor BMDM stimulated−/+ATP or nigericin (n=3), showing cGAS-STING dependence. (FIG. 14B) IB analysis of cGAS and STING in lysates of indicated BMDM. (FIG. 14C) Representative fluorescent microscopy images of LPS-primed BMDM pretreated−/+CsA (1 μM, 16 hrs), VBIT-4 (10 μM, 16 hrs) or FEN1-IN-4 (10 μM, 16 hrs) and stained for phospho-STING (Ser³⁶⁵) before or after ATP or nigericin stimulation. DAPI stains nuclei. Scale bar, 5 μm. (FIG. 14D) Percentages of cells from (FIG. 14C) with p-STING (Ser³⁶⁵) puncta. n=150 cells per group were analyzed in 3 independent experiments. (FIG. 14E) IB analysis of STING Ser³⁶⁵ phosphorylation in lysates and Casp1 p20 and IL-1β in supernatants of LPS-primed WT or mt-Ogg1Tg BMDM stimulated−/+ATP or nigericin (left) (n=3). Mitochondrial OGG1 IB in WT and mt-Ogg1Tg BMDM (right). (FIG. 14F) Relative p-STING (Ser³⁶⁵) to total STING amounts in cells from (FIG. 14E). Results in (FIG. 14A, FIG. 14D and FIG. 14F) are mean±s.d. *p<0.05; **p<0.01; ***p<0.001. Two-sided unpaired t-test.

FIG. 15: A schematic working model illustrating how Ox-mtDNA fragments escape to the cytosol to activate NLRP3Inf and STING.

### DETAILED DESCRIPTION

### Definitions

As it would be understood, the section or subsection headings as used herein is for organizational purposes only and are not to be construed as limiting or separating or both limiting and separating the subject matter described.

Throughout this disclosure, various technical publications, patents and published patent specifications are referenced by an identifying citation or a reference to a citation that immediately precedes the claims. The disclosures of these publications, patents and published patent specifications are hereby incorporated by reference into the present disclosure in their entireties to more fully describe the state of the art to which this invention pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M.J. MacPherson, B.D. Hames and G.R. Taylor eds. (1995)), Harlow and Lane, eds. (1988) Antibodies, a Laboratory Manual, and Animal Cell Culture (R.I. Freshney, ed. (1987)).

As used in the specification and claims, the singular form “a,” “an” and “the” include plural references unless the context clearly dictates otherwise. For example, the term “a cell” includes a plurality of cells, including mixtures thereof. As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human.

A “composition” as used herein, refers to an active agent, such as a compound as disclosed herein and a carrier, inert or active. The carrier can be, without limitation, solid such as a bead or resin, or liquid, such as phosphate buffered saline.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

The terms “oligonucleotide” or “polynucleotide” or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The term “contacting” means direct or indirect binding or interaction between two or more molecules or agents. A particular example of direct interaction is binding of an antibody to an antigen. A particular example of an indirect interaction is where one entity acts upon an intermediary molecule, which in turn acts upon the second referenced entity. Contacting as used herein includes the contacting in solution, in solid phase, in vitro, ex vivo, in a cell and in vivo in a tissue or subject. Contacting in vivo can be referred to as administering, or administration.

As used herein, an amino acid (aa) or nucleotide (nt) residue position in a sequence of interest “corresponding to” an identified position in a reference sequence refers to the residue position is aligned to the identified position in a sequence alignment between the sequence of interest and the reference sequence. Various programs are available for performing such sequence alignments, such as Clustal Omega and BLAST. In one aspect, equivalent polynucleotides, proteins, and corresponding sequences can be determined using BLAST (accessible at blast.ncbi.nlm.nih.gov/Blast.cgi, last accessed on Aug. 1, 2021).

It is to be inferred without explicit recitation and unless otherwise intended, that when the present disclosure relates to a polypeptide, amino acid sequence, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide, or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity, or at least about 85% homology or identity, or alternatively at least about 90% homology or identity, or alternatively at least about 95% homology or identity, or alternatively at least about 96% homology or identity, or alternatively at least about 97% homology or identity, or alternatively at least about 98% homology or identity, or alternatively at least about 99% homology or identity (in one aspect, as determined using the Clustal Omega alignment program) and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complementary sequence.

In some embodiments, a first sequence (nucleic acid sequence or amino acid) is compared to a second sequence, and the identity percentage between the two sequences can be calculated. In further embodiments, the first sequence can be referred to herein as an equivalent and the second sequence can be referred to herein as a reference sequence. In yet further embodiments, the identity percentage is calculated based on the full-length sequence of the first sequence. In other embodiments, the identity percentage is calculated based on the full-length sequence of the second sequence.

As used herein, the term “animal” refers to living multi-cellular vertebrate organisms, a category that includes, for example, mammals and birds. The term “mammal” includes both human and non-human mammals. Non-limiting examples of such include humans, bovines, canines, felines, murines, rats, and equines.

As used herein, a “FEN1 inhibitor” or “an inhibitor of human flap endonuclease-1 (FEN1)” refers to a compound or inhibitory oligonucleotide that reduces the activity of Flap endonuclease 1 when compared to a control, such as absence of the compound, oligo, or a compound with known inactivity. Wild type FEN1 protein is known in the art, e.g., as identified by NCBI Accession NP_004102. In embodiments, exemplary X-ray crystallographic structures for FEN1 are disclosed in PDB (Protein Data Bank) Nos. 5FV7, 5EOV, 3UVU, 3Q8K, 3Q8L, 3Q8M, and 1UL1. Non-limiting examples of FEN1 inhibitors include FEN-IN-4, and those identified in EP 3353154B1, CN106692155A, https://journals.plos.org/plosone/article?id=10.1371/journal.pone.0179278, https://www.sheffield.ac.uk/news/nr/blocking-enzymes-in-dna-damage-1.598305, and https://www.worldscientific.com/doi/abs/10.1142/S1793292021501411.

Several small molecule inhibitors have been reported in the literature, see Table 1, reproduced from Yang F, et al. (2022) Biomolecules, July 20;12 (7): 1007. doi: 10.3390/biom12071007. PMID: 35883563; PMCID: PMC9312813.

TABLE 1

| Compound | Chemical Structure |
|---|---|
| Compound #2 | 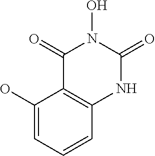 [SMILES: O=c1[nH]c2cccc(O)c2c(=O)n1O] |
| Compound #8 | 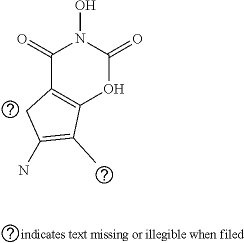 |
| Compound #16 | 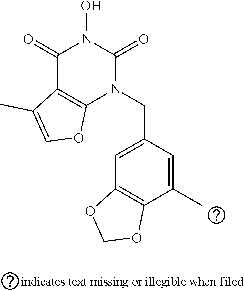 |
| Compound #20 | 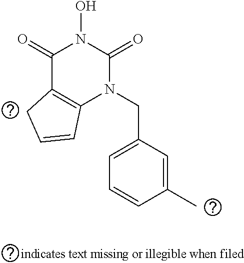 [SMILES: Cc1cccc(Cn2c3c(c(=O)n(O)c2=O)CC=C3)c1] |
| FEN1i | 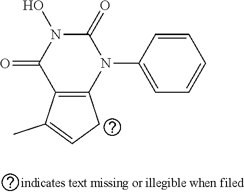 [SMILES: CC1=CCc2c1c(=O)n(O)c(=O)n2-c1ccccc1] |
| FEN1i #2 | 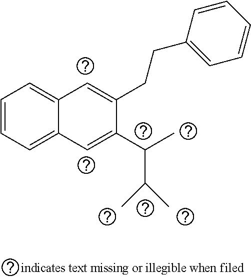 [SMILES: CC(C)C(C)c1cc2ccccc2cc1CCc1ccccc1] |

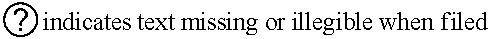

See also https://www.axonmedchem.com/product/3027 (accessed on May 3, 2023), for a structure of a commercially available inhibitor, reproduced below.

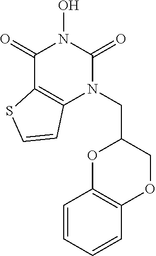 [SMILES: O=c1c2sccc2n(CC2COc3ccccc3O2)c(=O)n1O]

FEN1-IN-4 (FEN1 Inhibitor C2, JUN93587, Compound 2) is an inhibitor of human flap endonuclease-1 (hFEN1) that abrogated mtDNA fragmentation and cytosolic Ox-mtDNA and mtDNA release and inhibited alum-induced NLRP3 inflammasome-dependent IL-1β production in vivo and reduced neutrophil and monocyte infiltration. It is commercially available from Selleck Chem (https://www.selleckchem.com/products/fen 1-in-4.html), and Med Chem Express (https://www.medchemexpress.com/fen1-in-4.html), each accessed on May 3, 2023).

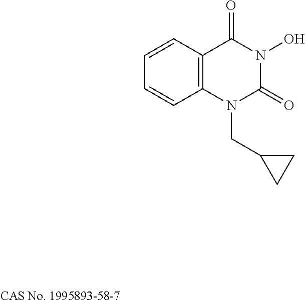 [SMILES: O=c1c2ccccc2n(CC2CC2)c(=O)n1O]

“mtDNA” intends mitochondrial DNA that are in the mitochondria of cells. They are known to encode essential protein subunits of the oxidative phosphorylation system. See, West and Shadel (2017) Nature Rev. Immunol. June 17 (6): 363-375.

As used herein, the term “inhibition of mtDNA fragmentation” intends an ability to inhibit, reduce or minimize the production of mtDNA fragmentation in a cell, tissue or organism as compared to a control or the absence of intervention in the system, tissue, or cell. Methods to measure mtDNA fragmentation are known in the art and described herein.

As used herein, the term “inhibition of cytostolic Ox-mtDNA” intends the ability to, inhibit, reduce or minimize the production of cytostolic Ox-mtDNA in a cell, tissue or organism as compared to a control or the absence of intervention in the system, tissue or cell.

As used herein, the term “inhibition of mtDNA release” intends an ability to inhibit the production of fragmented mtDNA in a cell or organism as compared to a control or the absence of intervention in the system or cell.

As used herein, the term “inhibition of Casp1 activation and IL-1β” intends an ability to inhibit the Casp1 activation and IL-1β production in a cell or organism as compared to a control or the absence of intervention in the system or cell.

As used herein, the term “administer” or “administration” or “administering” intends to mean delivery of a substance to a subject such as an animal or human. Administration can be in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and animals, treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and the target cell or tissue. Non-limiting examples of route of administration include intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmucosal, and inhalation.

An agent of the present disclosure can be administered for therapy by any suitable route of administration. It will also be appreciated that the optimal route will vary with the condition and age of the recipient, and the disease being treated.

As used herein, the term “a disease or condition associated with one or more of mtDNA fragmentation; cytostolic Ox-mtDNA; mtDNA release; or Casp1 activation and IL-1β production” intends any number of chronic diseases and metabolic pathologies, including obesity, type 2 diabetes, atherosclerosis, inflammatory bowel diseases, gout, and periodic fevers, as well as neurodegenerative disorders, including Parkinson's and Alzheimer's diseases (AD), atherosclerosis, NASH, asthma, inflammatory bowel disease, and melanoma. It one aspect, cancer such as melanoma is excluded from the group of disorders or conditions.

“Therapeutically effective amount” or “effective amount” of a drug or an agent refers to an amount of the drug or the agent that is an amount sufficient to obtain a pharmacological response such as passive immunity; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose and may occur only after administration of a series of doses. Thus, a therapeutically effective amount may be administered in one or more administrations.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

The down regulation of existing genes within the cell can be utilized. “Inhibiting expression” or “down regulating expression” is a process resulting in the decreased gene and corresponding protein expression. Reducing expression of a gene described herein can be done by a variety of method known in the art. Examples of which include the use of oligonucleotide-based strategies including interfering RNA technology, micro-RNA, siRNA, and vector based technologies including insertional mutagenesis, Cre-Lox deletion technology, double-stranded nucleic acid RNA/RNA, DNA/DNA, RNA/DNA, CRISPR, TALE, Zinc Finger technology and the like. All of these are non-limiting examples of inhibitory oligonucleotides.

### Modes For Carrying Out the Disclosure

NLRP3 activation is associated to many inflammatory and neurodegenerative diseases (e.g. Alzheimer's, Parkinson's, atherosclerosis, NASH, asthma, inflammatory bowel disease, melanoma, etc.). Oxidized mitochondrial DNA (Ox-mtDNA) leaves the mitochondria and into the cytosol of the cell and then binds to cytosolic NLRP3 inducing inflammasome activation. mtDNA fragments in the cytosol can also activate the cGAS-STING pathway. In fact, the Ox-mtDNA is cleaved by the endonuclease FEN1 to 500-650 bp fragments which then activate the NLRP3 inflammasome. Applicant identified FEN1 (human flap endonuclease-1), a thermostable DNA/RNA endonuclease, which cleaves 5′ DNA and RNA flaps from branched double stranded DNA substrates, creating a 4′ phosphate terminus, as a target for inhibitors that can suppress IL-1β production and mtDNA release in vivo. Applicant also show that the published inhibitor FEN1-IN-4 abolishes mtDNA fragmentation and cytosolic Ox-mtDNA and mtDNA release, which resulted in a dose-dependent inhibition of Casp1 activation and IL-1β, but not TNF secretion.

Mitochondrial DNA (mtDNA) escaping stressed mitochondria provokes inflammation via the cGAS-STING pathway and when oxidized (Ox-mtDNA) it binds cytosolic NLRP3, triggering inflammasome (Inf) activation. However, it is unknown how and in which form Ox-mtDNA exits stressed mitochondria in non-apoptotic macrophages. Applicant shows that diverse NLRP3Inf activators rapidly stimulate calcium uptake via the mitochondrial calcium uniporter (MCU) to open mitochondrial permeability transition pores (mPTP) that trigger VDAC oligomerization independently of mtDNA or reactive oxygen species that convert newly synthesized mtDNA to Ox-myDNA. Within mitochondria, Ox-mtDNA is repaired by DNA glycosylase OGG1 or cleaved by endonuclease FEN1 to 500-650 bp fragments that are released via mPTP- and VDAC-dependent channels to initiate NLRP3Inf activation. This pathway also leads to cGAS-STING activation and generation of pro-inflammatory extracellular mtDNA. Its improved understanding will facilitate development of new treatments for chronic inflammatory diseases, exemplified by FEN1 inhibitors that suppress IL-1β production and mtDNA release in mice.

### Therapeutic Methods

This disclosure provides a method in a mammalian cell for one or more, two or more, three or more, or all four of: 1) inhibition of mtDNA fragmentation; 2) inhibition of cytostolic Ox-mtDNA; 3) inhibition of mtDNA release; or 4) inhibition of Casp1 activation and IL-1β production, comprising, or consisting essentially of, or yet further consisting of contacting the mammalian cell with an effective amount of an inhibitor of human flap endonuclease-1 (FEN1), thereby inhibiting mtDNA fragmentation; inhibiting cytostolic Ox-mtDNA; inhibiting mtDNA release; or inhibiting of Casp1 activation and IL-1β production. In some embodiments, the inhibitor of human flap endonuclease-1 (FEN1) is a small molecule or an inhibitory oligonucleotide. In another aspect, the inhibitor of FEN1 is FEN-IN-4. These agents can be delivered in combination with a carrier, such as a pharmaceutically acceptable carrier. The cell can be any mammalian cell, non-limiting examples of such include a human cell, a murine cell, a rat cell, a canine cell, a feline cell, or a bovine cell. The contacting can be in vitro or in vivo. Methods to determine inhibition are known in the art and described herein.

This disclosure provides a method for inhibition of mtDNA fragmentation comprising, or consisting essentially of, or yet further consisting of contacting the mammalian cell with an effective amount of an inhibitor of human flap endonuclease-1 (FEN1), thereby inhibiting mtDNA fragmentation. In some embodiments, the inhibitor of human flap endonuclease-1 (FEN1) is a small molecule or an inhibitory oligonucleotide. In another aspect, the inhibitor of FEN1 is FEN-IN-4. These agents can be delivered in combination with a carrier, such as a pharmaceutically acceptable carrier. The cell can be any mammalian cell, non-limiting examples of such include a human cell, a murine cell, a rat cell, a canine cell, a feline cell, or a bovine cell. The contacting can be in vitro or in vivo. Methods to determine inhibition are known in the art and described herein.

This disclosure provides a method for inhibition of cytostolic Ox-mtDNA in a mammalian cell, comprising, or consisting essentially of, or yet further consisting of contacting the mammalian cell with an effective amount of an inhibitor of human flap endonuclease-1 (FEN1), thereby inhibiting cytostolic Ox-mtDNA; inhibiting mtDNA release. In some embodiments, the inhibitor of human flap endonuclease-1 (FEN1) is a small molecule or an inhibitory oligonucleotide. In another aspect, the inhibitor of FEN1 is FEN-IN-4. These agents can be delivered in combination with a carrier, such as a pharmaceutically acceptable carrier. The cell can be any mammalian cell, non-limiting examples of such include a human cell, a murine cell, a rat cell, a canine cell, a feline cell, or a bovine cell. The contacting can be in vitro or in vivo. Methods to determine inhibition are known in the art and described herein.

This disclosure provides a method for inhibition of mtDNA release in mammalian cell, comprising, or consisting essentially of, or yet further consisting of contacting the mammalian cell with an effective amount of an inhibitor of human flap endonuclease-1 (FEN1), thereby inhibiting mtDNA release. In some embodiments, the inhibitor of human flap endonuclease-1 (FEN1) is a small molecule or an inhibitory oligonucleotide. In another aspect, the inhibitor of FEN1 is FEN-IN-4. These agents can be delivered in combination with a carrier, such as a pharmaceutically acceptable carrier. The cell can be any mammalian cell, non-limiting examples of such include a human cell, a murine cell, a rat cell, a canine cell, a feline cell, or a bovine cell. The contacting can be in vitro or in vivo. Methods to determine inhibition are known in the art and described herein.

This disclosure provides a method in a mammalian cell for inhibition of Casp1 activation and IL-1β production, comprising, or consisting essentially of, or yet further consisting of contacting the mammalian cell with an effective amount of an inhibitor of human flap endonuclease-1 (FEN1), thereby inhibiting of Casp1 activation and IL-1β production. In some embodiments, the inhibitor of human flap endonuclease-1 (FEN1) is a small molecule or an inhibitory oligonucleotide. In another aspect, the inhibitor of FEN1 is FEN-IN-4. These agents can be delivered in combination with a carrier, such as a pharmaceutically acceptable carrier. The cell can be any mammalian cell, non-limiting examples of such include a human cell, a murine cell, a rat cell, a canine cell, a feline cell, or a bovine cell. The contacting can be in vitro or in vivo. Methods to determine inhibition are known in the art and described herein.

When practiced, the methods are useful to screen for or confirm an inhibitor of FEN1 having the same, similar, or opposite ability as the agents or compounds identified herein. In one aspect, the agents or compounds can be detectably labeled. Alternatively, they can be used to identify which inhibitor of FEN1 is best suited to treat a condition as disclosed herein. For example, one can screen for new agents or combination therapies by having two samples containing for example, the cells and the agent to be tested. The second sample contains a known inhibitor and cells to serve as a positive control. In a further aspect, several samples are provided and the agents or compounds (small molecules for example) are added to the system in increasing dilutions to determine the optimal dose that would likely be effective in treating a subject in the clinical setting. As is apparent to those of skill in the art, a negative control only containing the cell can be provided. In a further aspect, the DNA and/or agents are detectably labeled, for example with luminescent molecules that will emit a signal when brought into close contact with each other. The samples are contained under similar conditions for an effective amount of time for the agent to inhibit FEN1 as determined by the activity being assayed, e.g., for one or more, two or more, three or more, or all four of: 1) inhibition of mtDNA fragmentation; 2) inhibition of cytostolic Ox-mtDNA; 3) inhibition of mtDNA release; or 4) inhibition of Casp1 activation and IL-1ß production.

Further provide are methods for treating a disease or condition associated with mtDNA fragmentation; cytostolic Ox-mtDNA; mtDNA release; or Casp1 activation and IL-1β production in a subject in need thereof, comprising administering to the subject an effective amount of an inhibitor of human flap endonuclease-1 (FEN1), thereby treating the disease or condition associated with mtDNA fragmentation; cytostolic Ox-mtDNA; mtDNA release; or Casp1 activation and IL-1β in the subject. In some embodiments, the disease or condition is an inflammatory or a neurodegenerative disease, non-limiting examples of such include, Alzheimer's, disease, Parkinson's disease, atherosclerosis, NASH (a form of non-alcoholic fatty liver disease), asthma, inflammatory bowel disease, and melanoma, optionally excluding cancer such as melanoma is excluded from the group of disorders or conditions. In other aspects, the any number of chronic diseases and metabolic pathologies, including obesity, type 2 diabetes, atherosclerosis, inflammatory bowel diseases, gout, and periodic fevers, as well as neurodegenerative disorders, including Parkinson's and Alzheimer's diseases (AD), atherosclerosis, NASH, asthma, inflammatory bowel disease, and melanoma, optionally excluding cancer such as melanoma is excluded from the group of disorders or conditions.

The method can be practiced on an animal and provide a therapy for a pet or other animal or in a laboratory animal and used to test for personalized therapies, or new combination therapies. The administration of the inhibitor of FEN1 is an effective amount to treat the disease or condition. In one aspect, prevention is excluded from the term “treatment” in the context of this embodiment.

In some embodiments, the inhibitor of human flap endonuclease-1 (FEN1) is a small molecule or an inhibitory oligonucleotide. In another aspect, the inhibitor of FEN1 is FEN-IN-4. These agents can be delivered in combination with a carrier, such as a pharmaceutically acceptable carrier.

In aspects of this method, the subject is a mammal, e.g. a human patient, a murine, a rat, a canine, a feline, or a bovine.

The compositions of the present disclosure can be administered by parenteral (e.g., intramuscular, intraperitoneal, intravenous, ICV, intracisternal injection or infusion, subcutaneous injection, or implant), oral, by inhalation spray nasal, vaginal, rectal, sublingual, urethral (e.g., urethral suppository) or topical routes of administration (e.g., gel, ointment, cream, aerosol, etc.) and can be formulated in suitable dosage unit formulations containing conventional non-toxic pharmaceutically acceptable carriers, adjuvants, excipients, and vehicles appropriate for each route of administration.

After a determination that the disease or condition is present, a FEN-1 inhibitor alone or in combination with a carrier, can be administered.

Administration can be in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. An agent of the present invention can be administered for therapy by any suitable route of administration. It will also be appreciated that the preferred route will vary with the condition and age of the recipient, and the disease being treated. Thus, routes of administration applicable to the methods of the invention include intranasal, intramuscular, urethrally, intratracheal, subcutaneous, intradermal, topical application, intravenous, rectal, nasal, oral, inhalation, and other enteral and parenteral routes of administration. Routes of administration may be combined, if desired, or adjusted depending upon the agent and/or the desired effect. An active agent can be administered in a single dose or in multiple doses. Embodiments of these methods and routes suitable for delivery, include systemic or localized routes. In general, routes of administration suitable for the methods of the invention include, but are not limited to, direct injection, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the inhibiting agent. Where systemic delivery is desired, administration typically involves invasive or systemically absorbed topical or mucosal administration of pharmaceutical preparations.

Methods of administration of the active through the skin or mucosa include, but are not limited to, topical application of a suitable pharmaceutical preparation, transcutaneous transmission, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

In various embodiments of the methods of the invention, the inhibitor of FEN1 will be administered by inhalation, injection or orally on a continuous, daily basis, at least once per day (QD), and in various embodiments two (BID), three (TID), or even four times a day. Typically, the therapeutically effective daily dose will be at least about 1 mg, or at least about 10 mg, or at least about 100 mg, or about 200-about 500 mg, and sometimes, depending on the compound, up to as much as about 1 g to about 2.5 g.

Dosing of can be accomplished in accordance with the methods of the invention using capsules, tablets, oral suspension, suspension for intra-muscular injection, suspension for intravenous infusion, gel or cream for topical application, or suspension for intra-articular injection.

Dosage, toxicity and therapeutic efficacy of compositions described herein can be determined by standard pharmaceutical procedures in cell cultures or experimental animals, for example, to determine the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In some embodiments, an effective amount of a composition sufficient for achieving a therapeutic or prophylactic effect, ranges from about 0.000001 mg per kilogram body weight per administration to about 10,000 mg per kilogram body weight per administration. Suitably, the dosage ranges are from about 0.0001 mg per kilogram body weight per administration to about 100 mg per kilogram body weight per administration. Administration can be provided as an initial dose, followed by one or more “booster” doses. Booster doses can be provided a day, two days, three days, a week, two weeks, three weeks, one, two, three, six or twelve months after an initial dose. In some embodiments, a booster dose is administered after an evaluation of the subject's response to prior administrations.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

### Compositions

In another aspect, provided herein are compositions for use in the methods as disclosed herein, wherein the compositions contain a FEN1 inhibitor that further comprises at least one carrier, such as a pharmaceutically acceptable carrier or excipient. In one aspect, the composition further comprises a preservative or stabilizer.

Compositions, including pharmaceutical compositions comprising, consisting essentially of, or consisting of the virus can be in combination of other therapeutic agents can be manufactured by means of conventional mixing, dissolving, granulating, dragee-making levigating, emulsifying, encapsulating, entrapping, or lyophilization processes. These can be formulated in conventional manner using one or more physiologically acceptable carriers, diluents, excipients, or auxiliaries which facilitate processing of the combinations of compounds provided herein into preparations which can be used pharmaceutically.

In some embodiments, the pharmaceutical formulations described herein are administered to a subject by multiple administration routes, including but not limited to, parenteral, subcutaneous, oral, buccal, rectal, sublingual, or transdermal administration routes. In some cases, parenteral administration comprises, or consists essentially of, or yet further consists of, intravenous, subcutaneous, intramuscular, intracerebral, intranasal, intra-arterial, intra-articular, intradermal, intravitreal, intraosseous infusion, intraperitoneal, or intrathecal administration. In some instances, the pharmaceutical composition is formulated for local administration. In other instances, the pharmaceutical composition is formulated for systemic administration.

In some embodiments, the pharmaceutical formulations include, but are not limited to, lyophilized formulations, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pennsylvania 1975, Liberman, H.A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins1999).

In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as AVICEL®, dibasic calcium phosphate, dicalcium phosphate dihydrate, tricalcium phosphate, calcium phosphate, anhydrous lactose, spray-dried lactose, pregelatinized starch, compressible sugar, such as Di-PAC® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or AMIJEL®, or sodium starch glycolate such as PROMOGEL® or EXPLOTAB®, a cellulose such as a wood product, methylcrystalline cellulose, e.g., AVICEL®, AVICEL® PH101, AVICEL®PH102, AVICEL® PH105, ELCEMA® P100, EMCOCEL®, VIVACEL®, MING TIA®, and SOLKA-FLOC®, methylcellulose, croscarmellose, or a cross-linked cellulose, such as cross-linked sodium carboxymethylcellulose (AC-DI-SOL®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as VEEGUM® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (STEROTEX®), higher fatty acids and their alkali-metal and alkaline earth metal salts, such as aluminum, calcium, magnesium, zinc, stearic acid, sodium stearates, glycerol, talc, waxes, STEAROWET®, boric acid, sodium benzoate, sodium acetate, sodium chloride, leucine, a polyethylene glycol (e.g., PEG-4000) or a methoxypolyethylene glycol such as CARBOWAX™, sodium oleate, sodium benzoate, glyceryl behenate, polyethylene glycol, magnesium or sodium lauryl sulfate, colloidal silica such as SYLOID™, CAB-O-SIL®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium docusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, poly sorb ate-20 or TWEEN® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., PLURONIC® (BASF), and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

The pharmaceutical compositions for the administration can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition, each compound of the combination provided herein is included in an amount sufficient to produce the desired therapeutic effect. For example, pharmaceutical compositions of the present technology may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the combination of compounds can be formulated as solutions, gels, ointments, creams, suspensions, etc., as is well-known in the art.

Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the combination of compounds provided herein can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the combination of compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the present technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

In some embodiments, one or more compositions disclosed herein are contained in a kit. Accordingly, in some embodiments, provided herein is a kit comprising, consisting essentially of, or consisting of one or more compositions disclosed herein and instructions for their use.

### Dosage and Dosage Formulations

In some embodiments, the compositions are administered to a subject suffering from a condition as disclosed herein, such as a mammal or a human, either alone or as part of a pharmaceutically acceptable formulation, once a week, once a day, twice a day, three times a day, or four times a day, or even more frequently.

Administration of the composition or combination alone or in combination with the additional therapeutic agent and compositions containing same can be effected by any method that enables delivery to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens can be adjusted to provide the optimum desired response. For example, a single bolus can be administered, several divided doses can be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient can also be determined, as can the temporal requirements for administering each agent to provide a detectable therapeutic benefit to the patient. Accordingly, while certain dose and administration regimens are exemplified herein, these examples in no way limit the dose and administration regimen that can be provided to a patient in practicing the present disclosure.

It is to be noted that dosage values can vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration are well-known in the relevant art and would be understood to be encompassed by the skilled artisan once provided the teachings disclosed herein.

### Diagnostic Methods

In some embodiments, one or more of the methods described herein further comprise, or consists essentially of, or yet further consists of, a diagnostic step. In some instances, a sample is first obtained from a subject suspected of having a disease or condition described above. Exemplary samples include, but are not limited to, cell sample, tissue sample, tumor biopsy, liquid samples such as blood and other liquid samples of biological origin (including, but not limited to, peripheral blood, sera, plasma, ascites, urine, cerebrospinal fluid (CSF), sputum, saliva, bone marrow, synovial fluid, aqueous humor, amniotic fluid cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In some instances, the sample is a tumor biopsy. In some cases, the sample is a liquid sample, e.g., a blood sample. In some cases, the sample is a cell-free DNA sample.

Various methods known in the art can be utilized to determine the presence of a disease or condition described herein or to determine if the therapy has been successful or requires modification. Assessment of one or more biomarkers associated with a disease or condition, can be performed by any appropriate method. Expression levels or abundance can be determined by direct measurement of expression at the protein or mRNA level, for example by microarray analysis, quantitative PCR analysis, or RNA sequencing analysis. Alternatively, labeled antibody systems may be used to quantify target protein abundance in the cells, followed by immunofluorescence analysis, such as FISH analysis.

### Kits

Also provided is a kit comprising an inhibitor of FEN1 and instructions for use in the methods disclosed herein. The kit comprises, or alternatively consists essentially of, or yet further consists of one or more of the combination or composition and instructions for use. In a further aspect, the instruction for use provides directions to conduct any of the methods disclosed herein.

As amenable, these suggested kit components may be packaged in a manner customary for use by those of skill in the art. For example, these suggested kit components may be provided in solution or as a liquid dispersion or the like.

As is apparent to those of skill in the art, the aforementioned methods and compositions can be combined with other therapeutic composition and agents for the treatment or the disclosed diseases or conditions.

### EXPERIMENTAL

Persistent NLRP3 signaling underlies many chronic diseases and metabolic pathologies, including obesity, type 2 diabetes, atherosclerosis, inflammatory bowel diseases, gout, and periodic fevers, as well as neurodegenerative disorders, including Parkinson's and Alzheimer's diseases (AD) (Halle et al., 2008; Heneka et al., 2015; Lamkanfi and Dixit, 2012; Mangan et al., 2018). Thus, improved understanding of the NLRP3 inflammasome activation pathway may advance treatment development for diverse diseases.

NLRP3 inflammasome activation follows a “two-step” route: priming and activation. Priming is initiated by Toll-like receptors (TLRs), which sense pathogen (PAMPs) or danger (DAMPs) associated molecular patterns and trigger nuclear factor kappa-light-chain-enhancer of activated B cells (NF-κB)-induced NLRP3 and pro-IL-1β transcription. Activation entails NLRP3 inflammasome assembly, Casp1 activation and IL-1β maturation. Heretofore, it has been unclear how diverse stimuli, referred to as NLRP3 activators, which include adenosine triphosphate (ATP), pore-forming toxins and different microcrystals (Broz and Dixit, 2016; Latz et al., 2013), trigger the abrupt priming-to-activation transition. Mitochondria are key coordinators of this process (Gurung et al., 2015; Nakahira et al., 2011; Shimada et al., 2012; Zhong et al., 2019; Zhong et al., 2018; Zhong et al., 2016; Zhou et al., 2011). Moreover Applicant previously found that in addition to NF-κB activation, TLR engagement results in interferon regulatory factor-1 (IRF1)-dependent induction of cytidine monophosphate kinase 2 (CMPK2), a rate-limiting mitochondrial nucleotide kinase needed for initiation of mtDNA synthesis (Xian et al., 2021; Zhong et al., 2018). Before its compaction into nucleoids, newly-synthesized mtDNA is exposed to reactive oxygen species (ROS), produced upon loss of mitochondrial membrane potential triggered by along with calcium influx and potassium efflux (Gurung et al., 2015; Zhong et al., 2016). This results in generation of Ox-mtDNA that is released to the cytosol where it binds NLRP3 to trigger NLRP3 inflammasome assembly (Shimada et al., 2012; Xian et al., 2021; Zhong et al., 2018). However, the mechanisms accounting for Ox-mtDNA processing and exit from stressed, but still intact, mitochondria remained unknown (Murphy, 2018). Another target for cytosolic mtDNA is the DNA-sensing enzyme cyclic GMP-AMP (cGAMP) synthase (cGAS), which leads to activation of stimulator of interferon genes (STING) and production of type I interferons (IFN), which further amplify the inflammatory response (Ablasser and Chen, 2019; Barber, 2015; Chen et al., 2016). Although NLRP3 shows clear preference for 8-oxoguanine (8-oxoG) containing DNA (Zhong et al., 2018), cGAS senses any kind of dsDNA. While extramitochondrial mtDNA has been shown to activate cGAS-STING (Riley and Tait, 2020; Wu et al., 2021), this response has not been shown to be triggered by external stimuli, especially not NLRP3 inflammasome activators or “agonists”. Applicant now reveal how Ox-mtDNA is processed to smaller fragments that enter the cytosol to trigger NLRP3 inflammasome assembly and activation, as well as STING phosphorylation, which mediates subsequent IFN production by non-apoptotic macrophages (Chen et al., 2016; Liu et al., 2015). The same pathway also accounts for release of circulating cell-free (ccf) mtDNA, a systemic inflammatory mediator (Oka et al., 2012; Zhong et al., 2019).

### Base Excision Repair Inhibits NLRP3 Inflammasome Activation and Sterile Inflammation

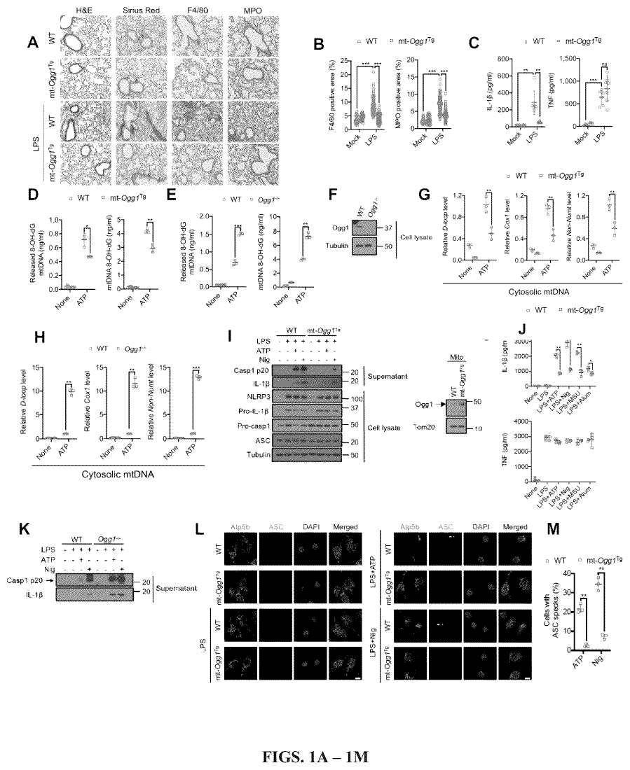

Ox-mtDNA promotes atherosclerosis, a disease in which 8-oxoG DNA-glycosylase 1 (OGG1), a base excision repair (BER) enzyme that removes 8-oxoG from damaged DNA, declines over time while NLRP3 inflammasome activity is augmented (Tumurkhuu et al., 2016). Genetic OGG1 deficiency increases risk of AD, whose pathogenesis is amplified by NLRP3 inflammasome activation (Heneka et al., 2015; Heneka et al., 2013), and high amounts of 8-oxoG containing DNA and reduced OGG1 activity are detected (Mao et al., 2007). In mice, OGG1 deficiency increases susceptibility to obesity and metabolic dysfunction (Sampath et al., 2012) and enhances type I IFN production and cutaneous manifestations in pristane induced lupus (Tumurkhuu et al., 2020). Although OGG1 is best-known for its nuclear function, it also localizes to mitochondria to maintain mtDNA integrity (de Souza-Pinto et al., 2001). Applicant wondered whether OGG1's anti-inflammatory effects are mitochondrially exerted and due to blunted NLRP3 inflammasome activation. Indeed, mitochondrially-targeted OGG1 transgenic (mt-Ogg1Tg) mice (Wang et al., 2011) were resistant to alum-induced NLRP3 inflammasome and IL-1β-dependent peritonitis (Figures S1A and S1B). Alum-challenged mt-Ogg1Tg mice exhibited reduced IL-1β and neutrophil and monocyte infiltration relative to wildtype (WT) counterparts, with little change in tumor necrosis factor (TNF). The NLRP3 inflammasome also instigates acute respiratory distress syndrome (ARDS), in which the lung parenchyma and resident immune cells generate IL-1β and IL-18 to drive pulmonary inflammation (Grailer et al., 2014; Xian et al., 2021). Notably, mt-Ogg1Tg mice were resistant to LPS-induced ARDS, displaying reduced pulmonary vascular endothelium damage, interstitial edema, macrophage and neutrophil infiltration alveolar wall thickening and collagen deposition (FIG. 1A and FIG. 1B). Fittingly, mt-Ogg1Tg mice had less IL-1β in bronchoalveolar lavage fluid (BALF) but no change in TNF secretion (FIG. 1C).

To understand how mtOGG1 inhibits IL-1β-driven inflammation, Applicant employed bone marrow derived macrophages (BMDM). ATP-challenged mt-Ogg1Tg BMDM had less 8-oxoG-containing Ox-mtDNA in cytoplasm and mitochondria than WT counterparts (FIG. 1D), whereas ATP-challenged Ogg1−/−-macrophages had more Ox-mtDNA in both compartments (FIG. 1E, FIG. 1F, FIG. 8C and FIG. 8D). Cytosolic mtDNA was also lower in mt-Ogg1Tg (FIG. 1G), and higher in Ogg1−/− (FIG. 1H and FIG. 8E) macrophages that were ATP challenged. mtOGG1 overexpression or lack thereof had no effect on the LPS-stimulated mtDNA increase (FIG. 8F-FIG. 8H), implying that mtOGG1 interferes with mtDNA oxidation, but not synthesis. Importantly, mtOGG1 inhibited Casp1 activation and pro-IL-1β processing in LPS-primed BMDM stimulated with ATP, nigericin, monosodium urate (MSU), or alum (FIG. 1I, 1J and FIG. 8I), whereas Ogg1 ablation had the opposite effect (FIG. 1K, FIG. 8J and FIG. 8 K). OGG1 had no effect on TNF secretion or pro-IL-1β and NLRP3 inflammasome component expression (FIG. 1I, FIG. 1J and FIG. 8L). ASC speck formation, reflecting inflammasome assembly (Dick et al., 2016), was also inhibited in mt-Ogg1Tg BMDM (FIG. 1L and FIG. 1M). These results reinforce the notion that Ox-mtDNA promotes NLRP3 inflammasome activation and is a target for the anti-inflammatory activity of mtOGG1. Although mtOGG1 inhibited and shOGG1 enhanced cytochrome C release and apoptotic activation of Casp3 and Casp9 (FIGS. 8M-8T), consistent with the notion that mtDNA damage triggers cardiomyocyte apoptosis which is attenuated by mtOGG1 (Ricci et al., 2008), NLRP3 inflammasome activators did not trigger apoptosis and apoptosis inducers did not activate NLRP3 inflammasome (see below).

### mPTP Opening and VDAC Oligomerization Enable Ox-mtDNA Leakage

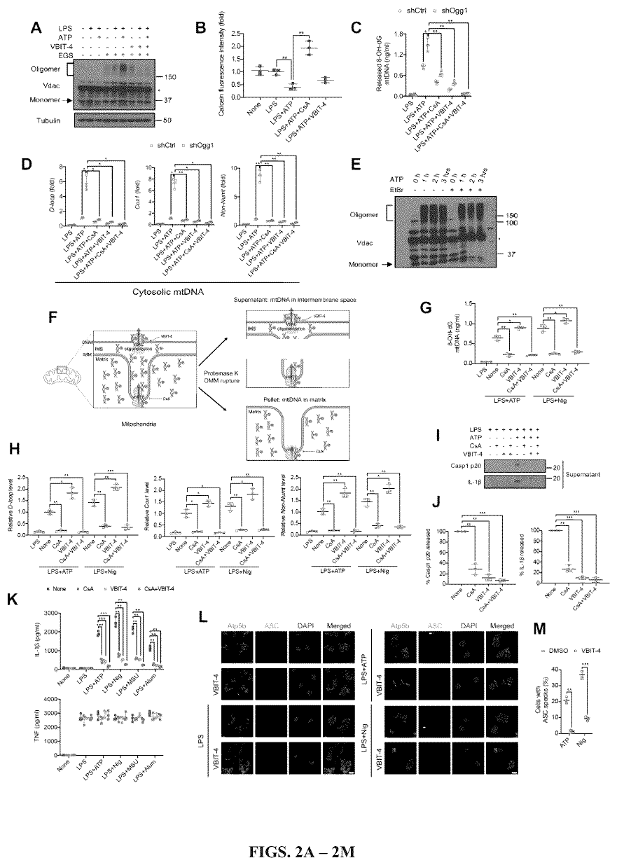

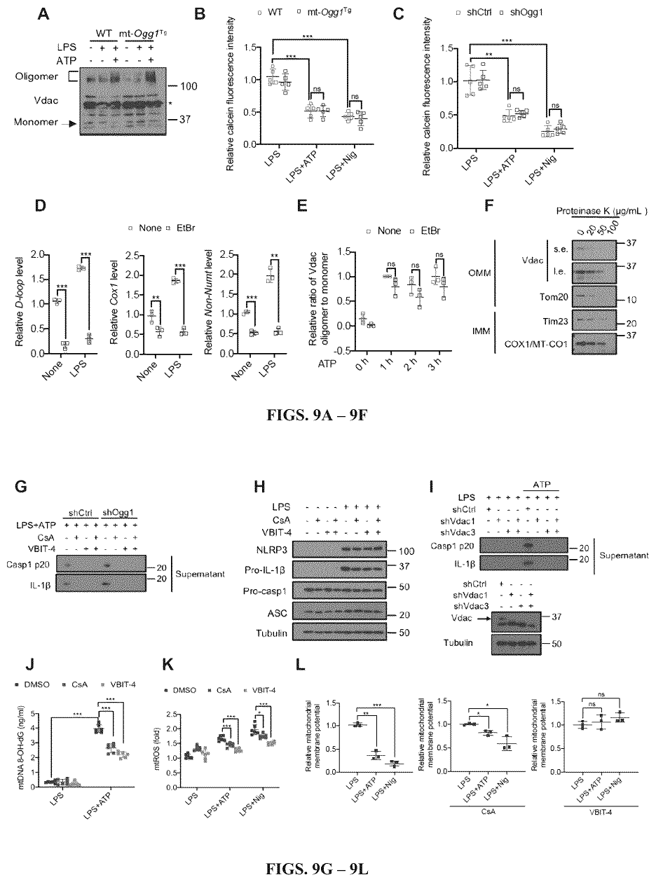

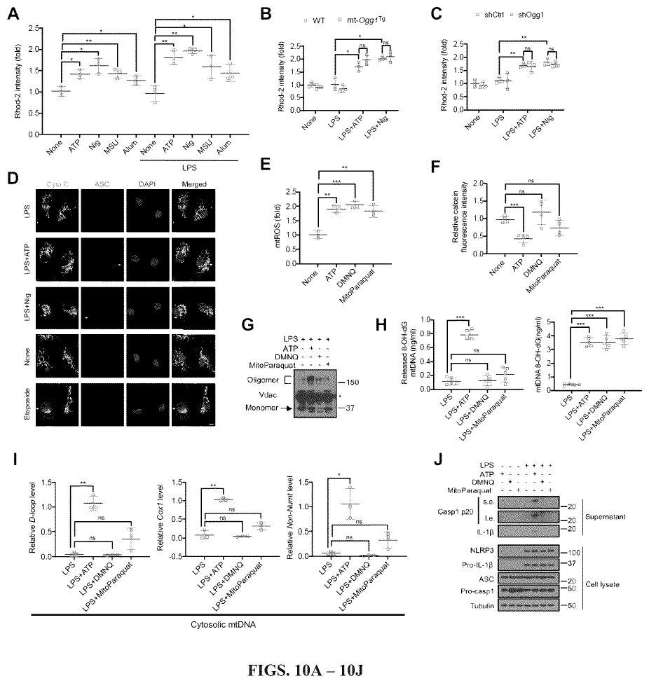

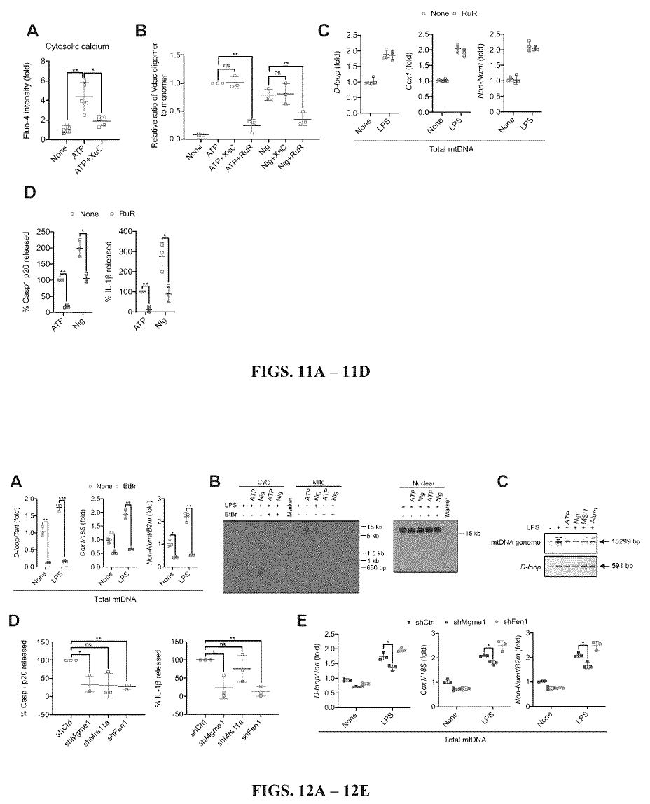

To activate NLRP3 inflammasome, Ox-mtDNA must exit mitochondria through an unknown pore and reach the cytosol (Murphy, 2018). Applicant examined the role of voltage-dependent anion channel (VDAC), recently reported to mediate mtDNA leakage in mitochondrial endonuclease g deficient (Endog−/−)-fibroblasts, which exhibit constitutive oxidative stress and VDAC oligomerization (Kim et al., 2019). In LPS-primed BMDM, VDAC oligomerization was induced by ATP challenge, indicating channel opening, and blocked by VBIT-4, a VDAC1 oligomerization inhibitor (FIG. 2A). VDAC resides in the outer mitochondrial membrane (OMM), but for mtDNA to exit via VDAC, it must pass through the inner mitochondrial membrane (IMM), where the poorly defined mitochondrial permeability transition pore (mPTP) is located (Perez-Trevino et al., 2020). Although the link between mPTP and VDAC remains contentious, mPTP was also suggested to be involved in mtDNA release and subsequent cGAS-STING activation in fibroblasts overexpressing TDP-43, a protein associated with amyotrophic lateral sclerosis (Yu et al., 2020). Importantly, ATP challenge induced mPTP opening, suggested by markedly decreased calcein fluorescence (FIG. 2B). The mPTP inhibitor cyclosporine A (CsA) that binds cyclophilin D (CyD), which mediates Ca2+-stimulated mPTP opening (Broekemeier et al., 1989), blocked the decrease in calcein fluorescence and increased it above baseline (FIG. 2B), suggesting low basal mPTP opening. However, the VDAC oligomerization inhibitor VBIT-4 had little effect on calcein fluorescence. Applicant therefore postulated that mPTP opening precedes VDAC oligomerization and enables Ox-mtDNA leakage to the cytosol. Although mtDNA oxidation was suggested to trigger its leakage (Kim et al., 2019; Perez-Trevino et al., 2020), mtOGG1 overexpression or ablation had no effect on VDAC oligomerization (FIG. 9A) or mPTP opening (FIG. 9B and FIG. 9C), suggesting that mitochondrial channel opening is not coupled to mtDNA oxidation (see more below). Indeed, inhibition of mPTP opening with CsA reduced the cytosolic pools of Ox-mtDNA by 60±6% and mtDNA by 30-40%, and VDAC oligomerization inhibition with VBIT-4 reduced cytosolic Ox-mtDNA by 81±4% and mtDNA by 50-60%, even in Ogg1-silenced BMDM (FIG. 2C and FIG. 2D). These results may explain how CsA inhibits NLRP3 inflammasome activation (Elliott and Sutterwala, 2015). In contrast to a previous report (Kim et al., 2019), mtDNA depletion with ethidium bromide (EtBr) did not block VDAC oligomerization (FIG. 2E, FIG. 9D and FIG. 9E). Thus, VDAC oligomerization and opening do not require mtDNA.

To dissect the roles of VDAC oligomerization and mPTP in mtDNA escape, Applicant investigated whether Ox-mtDNA and mtDNA were trapped in the mitochondrial intermembrane space (IMS) of VBIT-4 treated BMDM. Due to differential proteinase K sensitivity (Xiao et al., 2015), OMM proteins (VDAC and Tom20) were degraded when purified BMDM mitochondria were incubated with 20 μg/mL proteinase K for 30 min, while IMM proteins (Tim23 and COX1/MT-CO1) were not (FIG. 9F), implying that the OMM was ruptured and the IMM remained intact. Using this method (FIG. 2F), Applicant measured relative Ox-mtDNA and mtDNA amounts in the IMS. The amounts of Ox-mtDNA (FIG. 2G) and different regions of the mtDNA genome (FIG. 2H) within the IMS were greatly reduced after blocking mPTP opening with CsA but were elevated after inhibition of VDAC oligomerization with VBIT-4. Importantly, the CsA effect was dominant to that of VBIT-4, indicating that Ox-mtDNA first exited the IMM via an mPTP-dependent pore and then reached the cytoplasm through VDAC channels. Both CsA and VBIT-4 inhibited NLRP3 inflammasome activation in WT and Ogg1−/−-BMDM, without affecting NLRP3, pro-IL-1β, pro-casp1 and ASC expression or TNF secretion (FIGS. 2I-2K, FIG. 9G and FIG. 9H). Vdac1 or Vdac3 silencing also prevented Casp1 activation and mature IL-1β secretion (FIG. 9I), and ASC speck formation was inhibited by VBIT-4 (FIG. 2L and FIG. 2M). CsA and VBIT-4 reduced mtDNA oxidation by 33±7.5% and 45±5.0%, respectively (FIG. 9J), presumably due to a modest decline in mtROS production (FIG. 9K). These results and those of the proteinase K experiment suggest that despite the decline in mtROS, CsA and VBIT-4 mainly interfere with mtDNA cytosolic escape. Moreover, BMDM incubated with CsA, or VBIT-4 abrogated mitochondrial depolarization upon NLRP3 activator challenge (FIG. 9L), suggesting that mPTP and VDAC opening equilibrate proton gradients and lead to uncoupling which enhances mtROS production.

### Ca²⁺ Overload Initiates mPTP Opening and Subsequent VDAC Oligomerization

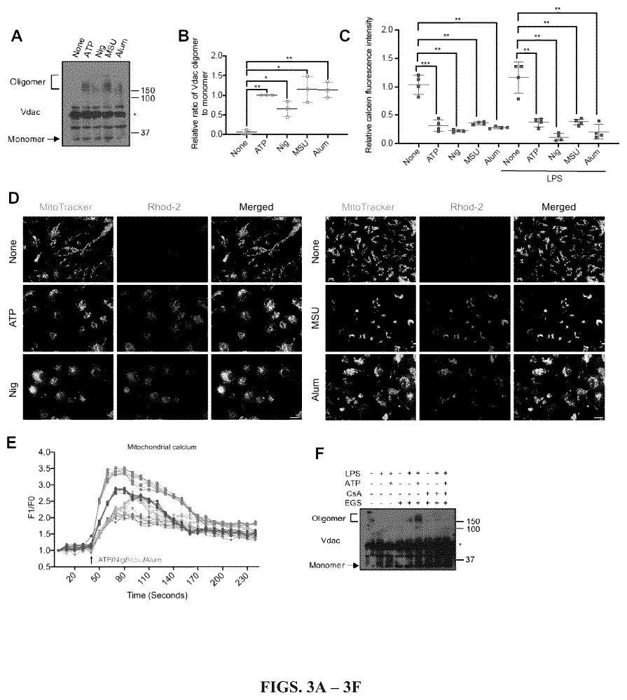

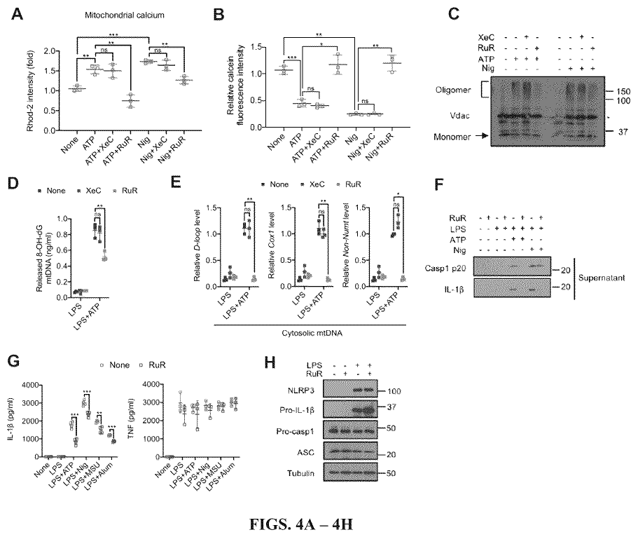

BMDM exposure to nigericin, MSU and alum, even without LPS priming, also triggered VDAC oligomerization (FIG. 3A and FIG. 3B) and mPTP opening (FIG. 3C), suggesting these are common events preceding NLRP3 inflammasome activation. To identify the trigger for mPTP opening, Applicant examined [Ca²⁺]m influx, because both calcium overload and ROS were suggested to drive mPTP opening (Bernardi et al., 2006). Rhod-2 (a dye that fluoresces in response to mitochondrial Ca²⁺ binding) fluorescence in BMDM stimulated with different NLRP3 activators, with or without LPS priming, was strongly elevated (FIG. 3D and FIG. 10A). A rapid [Ca²⁺]m spike was detected within 30-50 sec of NLRP3 agonist challenge (FIG. 3E), which may be the earliest mitochondrial response to NLRP3 inflammasome activators. mtOGG1 overexpression or deficiency had no impact on [Ca²⁺]m uptake amplitude (FIG. 10B and FIG. 10C). Notably, inhibition of mPTP opening with CsA suppressed VDAC oligomerization (FIG. 3F), indicating that mPTP opening triggered VDAC oligomerization. No cytochrome C release was observed during NLRP3 inflammasome activation and etoposide, which induced cytochrome C release, did not trigger ASC speck formation (FIG. 10D), suggesting mPTP-VDAC opening is apoptosis-independent. This fits well with the notion that apoptosis is linked to formation of OMM-resident BAX and BAK oligomerization macropores, which cause mPTP-independent IMM herniation to release mtDNA for cGAS-STING activation (McArthur et al., 2018; Riley et al., 2018; Wu et al., 2021). To examine mtROS involvement in NLRP3 agonist-induced channel opening and consequent Ox-mtDNA release, Applicant employed the mtROS generators DMNQ and mitoParaquat. Although producing as much mtROS as ATP (FIG. 10E), neither compound induced mPTP opening (FIG. 10F), VDAC oligomerization (FIG. 10G) and consequent Ox-mtDNA (FIG. 10H) and mtDNA (FIG. 10I) cytosolic release. While mtDNA oxidation was augmented (FIG. 10H), neither mtROS generator activated NLRP3 inflammasome (FIG. 10J).

### MCU Mediates Mitochondrial Ca²⁺ Uptake to Trigger mPTP-VDAC Channel Opening

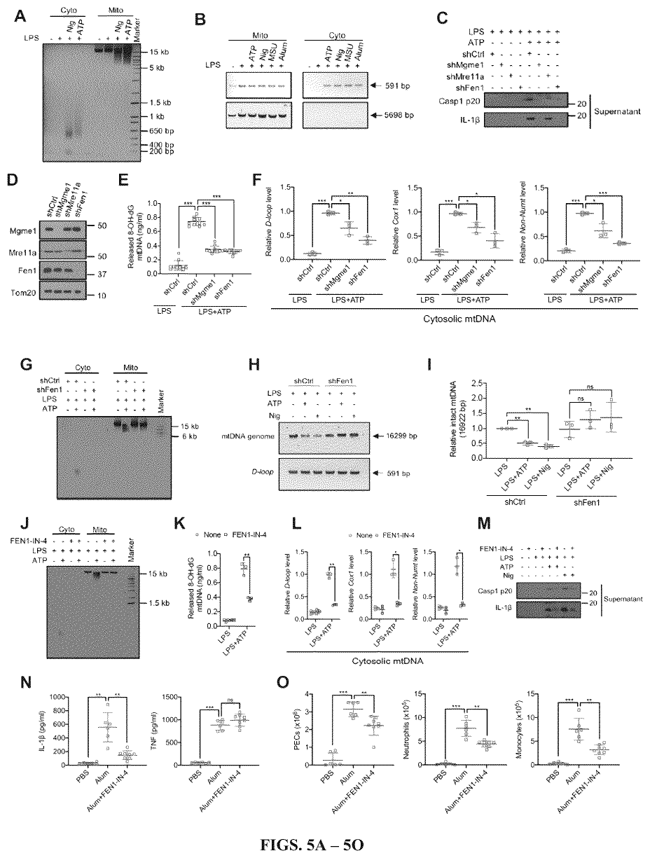

Ca²⁺ from the endoplasmic reticulum (ER), the major intracellular Ca²+ store, can be transferred via inositol 1,4,5-trisphosphate receptor (IP3R) to mitochondria (Rizzuto et al., 1998). Whereas the OMM is permeable to ions and small solutes, Ca²⁺ entry via the ion impermeable IMM is mediated by the mitochondrial calcium uniporter (MCU) complex (Baughman et al., 2011). To validate [Ca²⁺]m uptake as the trigger of mPTP opening, Applicant used xestospongin C (XeC), an IP3R antagonist and ruthenium red (RuR), a MCU inhibitor. Whereas RuR disrupted the ATP- or nigericin-induced [Ca²⁺]m pulse (FIG. 4A), XeC had no effect, although it decreased cytosolic Ca²⁺ (FIG. 11A). Importantly, RuR blocked mPTP opening (FIG. 4B) and VDAC oligomerization (FIG. 4C and FIG. 11B), reducing cytosolic Ox-mtDNA (FIG. 4D) and cytoplasmic mtDNA release (FIG. 4E). RuR did not inhibit LPS-stimulated mtDNA synthesis (FIG. 11C), but it suppressed Casp1 activation and mature IL-1β secretion (FIG. 4F, FIG. 4G and FIG. 11D), with no effect on NLRP3 inflammasome component expression (FIG. 4H) or TNF secretion (FIG. 4G).

### FEN1-Mediated mtDNA Fragmentation Precedes Cytosolic Escape and Licenses NLRP3 Inflammasome Activation

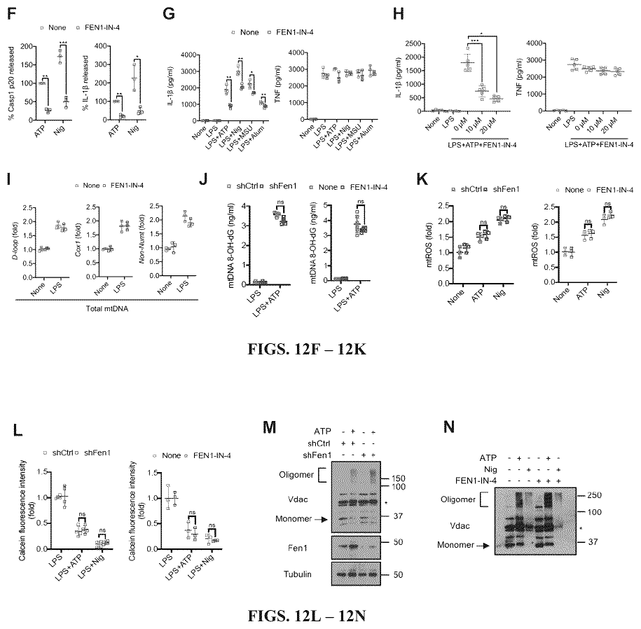

mtDNA is ˜16.3 kb in length and compacted by TFAM into a matrix-attached nucleoid that cannot pass through either mPTP or VDAC pores (Garcia and Chavez, 2007; Wu et al., 2021; Zhong et al., 2019). Although it has been proposed that entire nucleoids are extruded into the cytosol to activate cGAS-STING (White et al., 2014; Zhong et al., 2019), other studies using isolated mitochondria or liposomes have shown that only mtDNA fragments <700 bp are released upon mPTP opening (Garcia and Chavez, 2007). To address this issue, Applicant extracted DNA from mitochondria and cytosol of LPS-primed BMDM challenged with either nigericin or ATP and separated it by gel electrophoresis. LPS priming increased the amount of mtDNA, which was partially degraded after nigericin or ATP exposure (FIG. 5A). Although the majority of mtDNA remaining within mitochondria was >5 kb, nigericin and ATP treatments led to appearance of cytoplasmic DNA at 500-650 bp in length (FIG. 5A). This DNA was mitochondrially derived because it was absent in EtBr-treated BMDM (FIG. 12A and FIG. 12B). Nuclear DNA degradation was not observed, further implying that NLRP3 activators do not trigger apoptosis. Applicant PCR amplified 591 bp or 5698 bp mtDNA stretches from the mitochondrial and cytosolic DNA pools of LPS-primed BMDM challenged with different NLRP3 activators. Although LPS increased the amounts of both DNA stretches in the mitochondrial pool, only the shorter DNA could be amplified from the cytosol and only after exposure to NLRP3 activators (FIG. 5B). PCR amplification revealed that amounts of the entire mitochondrial genome (16299 bp) were higher after LPS priming, but lower after NLRP3 agonist challenge (FIG. 12C).

Searching for mitochondrial nucleases accounting for mtDNA cleavage, Applicant found that silencing of mitochondrial genome maintenance exonuclease 1 (Mgme1) or flap structure-specific endonuclease 1 (Fen1), but not meiotic recombination 11 homolog A (Mre11a), all of which participate in mtDNA metabolism (Fontana and Gahlon, 2020), attenuated NLRP3 inflammasome activation (FIG. 5C, FIG. 5D and FIG. 12D). Accordingly, Mgme1 or Fen1 silencing inhibited cytosolic Ox-mtDNA and mtDNA release (FIG. 5E and FIG. 5F). Whereas silencing of Mgme1, a component of the mitochondrial replisome (Matic et al., 2018), reduced mtDNA amounts in LPS-primed cells, Fen1 silencing did not (FIG. 12E). Importantly, Fen1 silencing inhibited mtDNA fragmentation and cytosolic release (FIG. 5G), and attenuated mtDNA integrity loss in ATP or nigericin challenged BMDM (FIG. 5H and FIG. 5I). The FEN1 inhibitor FEN1-IN-4 (Exell et al., 2016), also abrogated mtDNA fragmentation and cytosolic Ox-mtDNA and mtDNA release (FIGS. 5J-5L), resulting in dose-dependent inhibition of Casp1 activation and IL-1β, but not TNF, secretion (FIG. 5M and FIGS. 12F-12H). FEN1-IN-4 did not affect LPS-stimulated mtDNA synthesis (FIG. 12I) and like Fen1 shRNA, had no effect on Ox-mtDNA generation or mtROS production (FIG. 12J and FIG. 12K). Moreover, FEN1 did not affect mPTP opening (FIG. 12L) or VDAC oligomerization (FIG. 12M and FIG. 12N), suggesting that mtDNA cleavage and pore opening are not coupled. Notably, FEN1-IN-4 inhibited alum-induced NLRP3 inflammasome-dependent IL-1β production in vivo and reduced neutrophil and monocyte infiltration, without an effect on TNF secretion (FIG. 5N and FIG. 5O).

### mt-OGG1 and FEN1-IN-4 Restrain Extracellular mtDNA Release and Paracrine Inflammation

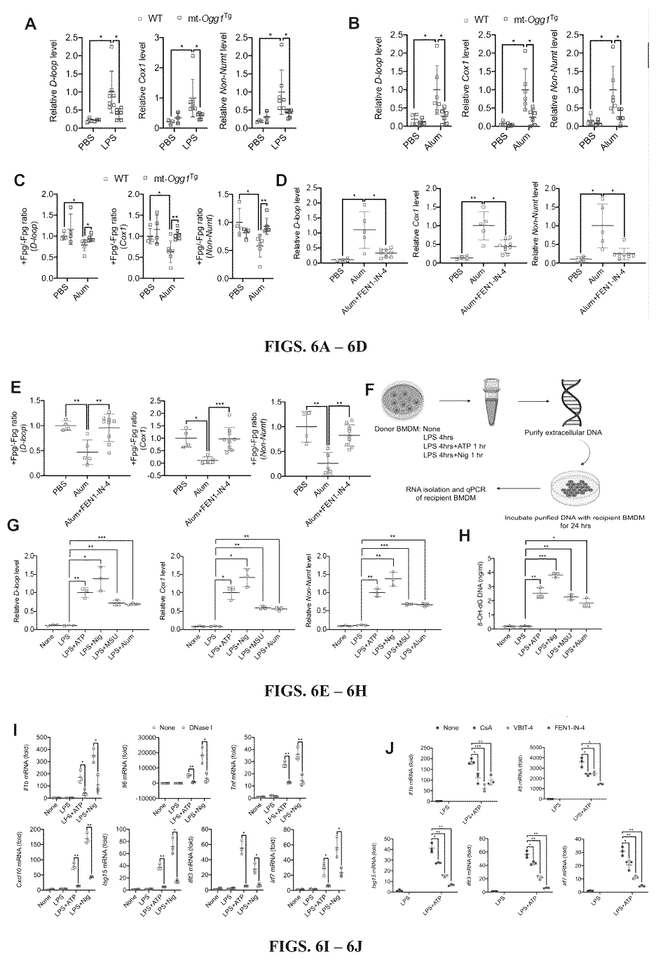

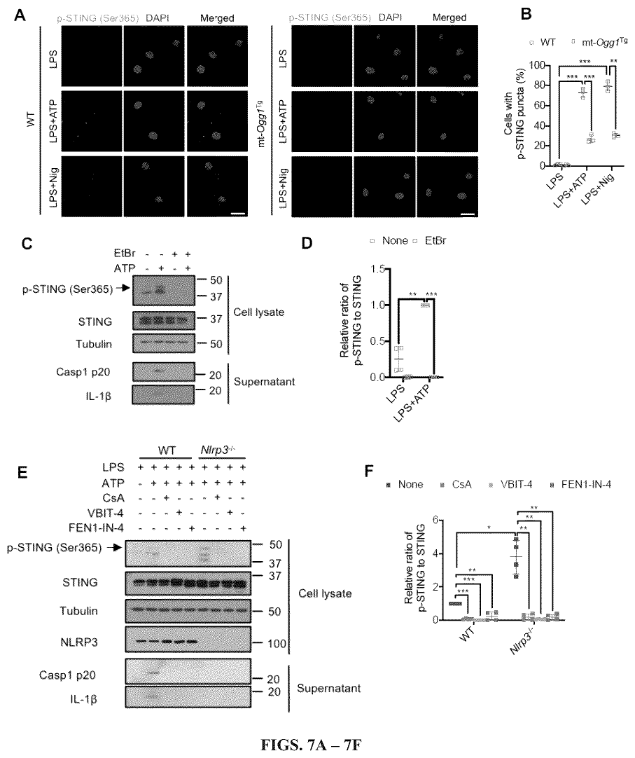

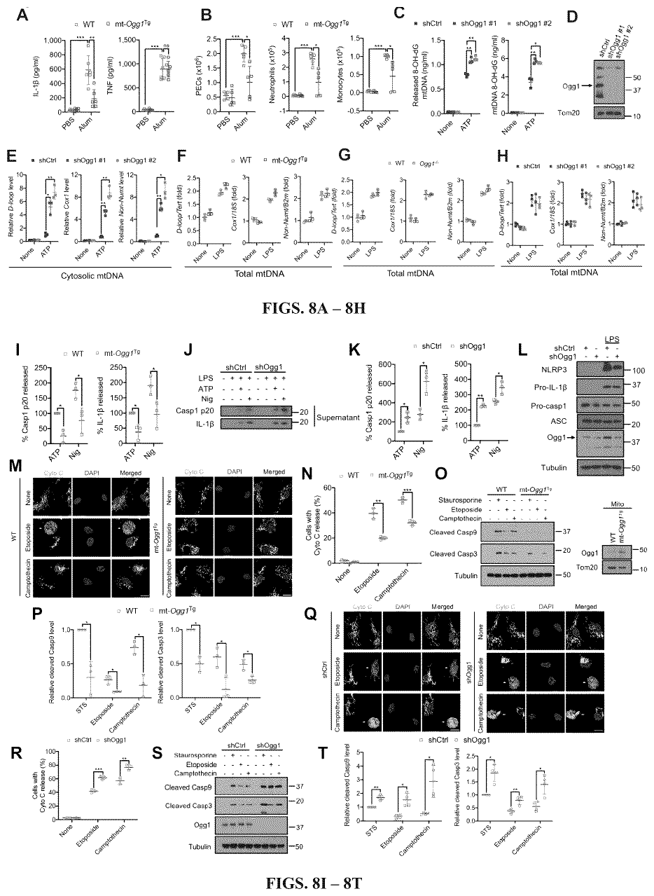

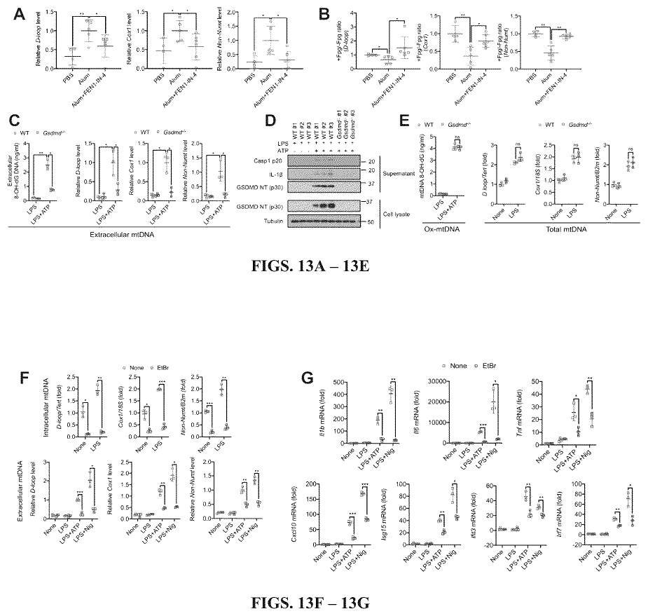

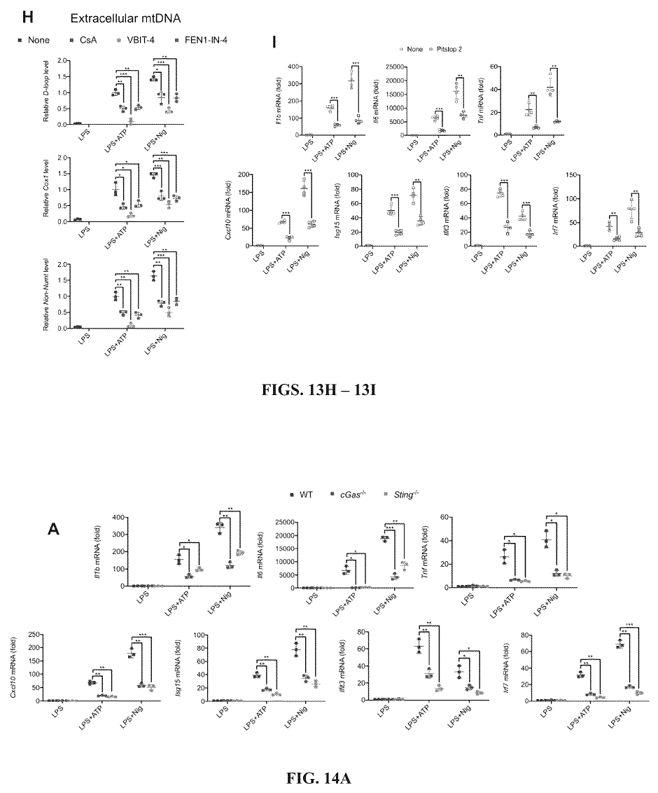

Ccf mtDNA present in body fluids, including plasma and serum (Duvvuri and Lood, 2019; Lam et al., 2004; Zhang et al., 2010; Zhong et al., 2000), contributes to arthritis, AD, heart failure, nonalcoholic steatohepatitis (NASH), and atherosclerosis (Desler et al., 2018; Oka et al., 2012; Tumurkhuu et al., 2016; Yu et al., 2019; Zhong et al., 2019). BALF of LPS-challenged mice undergoing ARDS contained elevated ccf-mtDNA amounts that were reduced in mt-Ogg1Tg mice (FIG. 6A). Cell-free peritoneal exudates isolated 4 hrs after alum challenge also contained more ccf-mtDNA than exudates from PBS-injected mice, and this was reduced in mt-Ogg1Tg mice (FIG. 6B). To detect oxidative lesions in ccf-mtDNA, Applicant employed formamidopyrimidine DNA glycosylase (Fpg), a bacterial 8-oxoG glycosylase and also an apurinic and apyrimidinic (AP) lyase, to cleave oxidized purines and remove the AP site, leaving a 1 base gap which blocks PCR amplification (Croteau and Bohr, 1997; Vartanian et al., 2017). Alum-induced ccf-mtDNA contained oxidative lesions and that was reduced by mt-OGG1 (FIG. 6C). Importantly, FEN1-IN-4 inhibited ccf-mtDNA and Ox-mtDNA secretion into peritoneal exudates isolated 4 and 16 hrs post-challenge (FIG. 6D, FIG. 6E, FIG. 13A and FIG. 13B), demonstrating that FEN1-mediated mtDNA fragmentation is needed for ccf-mtDNA generation.

To examine the immunogenic effect of secreted mtDNA, Applicant isolated cell-free Ox-mtDNA released by BMDM challenged with NLRP3 activators (FIGS. 6F-6H). Of note, gasdermin D deficient BMDM (Gsdmd^{-/-}) released much less Ox-mtDNA but generated as much Ox-mtDNA as WT BMDM (FIGS. 13C-13E). When applied to naïve macrophages, cell-free mtDNA induced NF-κB-dependent cytokine mRNAs, Il1b, Il6 and Tnf, and IFN-stimulated genes (ISGs), Cxcl10, Isg15, Ifit3 and Irf7 in a DNase I sensitive manner (FIG. 6I). EtBr pretreatment greatly reduced cell-free mtDNA amounts and immunostimulatory activity (FIG. 13F and FIG. 13G), validating its mitochondrial origin. CsA, VBIT-4 and FEN1-IN-4 also inhibited cell-free mtDNA release (FIG. 13H) and immunostimulatory activity (FIG. 6J). To understand how extracellular mtDNA enters macrophages, Applicant used Pitstop2, an inhibitor of clathrin-mediated endocytosis (von Kleist et al., 2011) and found it to diminish cytokine mRNA induction by extracellular mtDNA (FIG. 13I).

### The Mitochondrial NLRP3 Inflammasome activation Pathway Leads to STING Activation

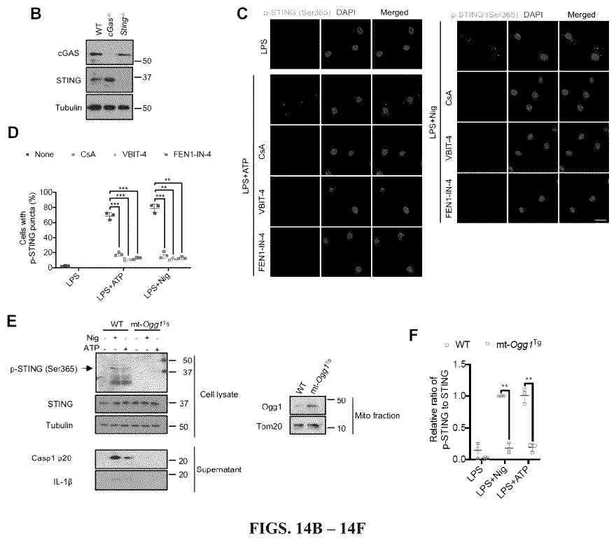

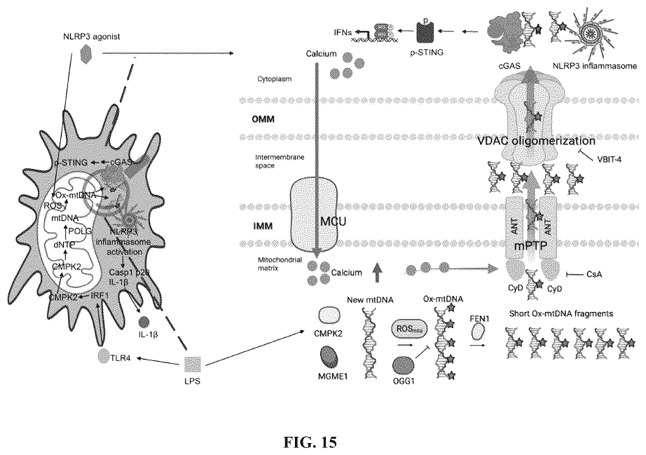

cGAS or STING ablation also blunted cytokine and ISG mRNA induction by extracellular mtDNA (FIG. 14A and FIG. 14B). Importantly, cytosolic release of Ox-mtDNA in LPS-primed BMDM stimulated with ATP or nigericin induced rapid formation of Ser³⁶⁵ phosphorylated STING puncta, which are essential for interferon regulatory factor-3 (IRF3) binding and activation (Chen et al., 2016; Liu et al., 2015). That was diminished by mtOGG1 expression and CsA, VBIT-4 and FEN1-IN-4 treatments (FIG. 7A, FIG. 7B, FIG. 14C and FIG. 14D). ATP-induced STING Ser³⁶⁵ phosphorylation was also detected by immunoblot analysis and was strictly dependent on mtDNA as it was undetectable in EtBr-treated BMDM (FIG. 7C and FIG. 7D). Nigericin also led to immunoblot detectable STING Ser³⁶⁵ phosphorylation and the response to it and ATP were abolished in mt-Ogg1Tg BMDM (FIG. 14E and FIG. 14F). Consistent with a report that inflammasome activation triggers Casp1-mediated cleavage of cGAS and thereby inhibits DNA virus induced cGAS-STING signaling (Wang et al., 2017), Applicant observed that Nlrp3−/−BMDM, in which Casp1 is not activated, exhibited enhanced ATP-induced STING Ser³⁶⁵ phosphorylation, that was completely blocked by CsA, VBIT-4 or FEN1-IN-4 (FIG. 7E and FIG. 7F), highlighting that Ox-mtDNA cleaved by FEN1 and released via mPTP-VDAC channels, licenses both NLRP3 inflammasome and cGAS-STING activation in cytosol.

### Discussion

In addition to their key role in cell survival and death, mitochondria have emerged as central regulators of inflammation (Gurung et al., 2015; Riley and Tait, 2020; Wu et al., 2021; Zhong et al., 2019). Mitochondria and NLRP3 inflammasome intersect at multiple facets and in different diseases (Broz and Dixit, 2016; Grailer et al., 2014; Gurung et al., 2015; Lamkanfi and Dixit, 2012; Latz et al., 2013). Applicant's results further establish mitochondria as primary targets for diverse NLRP3 activators, including ATP, a ligand for the purinergic receptor P2X7, nigericin, an antibiotic and an H+-K+ antiporter, and the membrane damaging microcrystals MSU and alum (Elliott and Sutterwala, 2015). Importantly, mitochondria play a key role in “two-step” NLRP3 inflammasome activation, as CMPK2-dependent mtDNA synthesis during “priming” couples with mtROS generation during “activation” to produce Ox-mtDNA, that enters the cytosol to trigger NLRP3 inflammasome activation (Zhong et al., 2018). Cytosolic mtDNA, containing non-oxidized and oxidized bases, also activates the cGAS-STING pathway, which unlike NLRP3, has no preference for oxidized DNA, having evolved to provide protection from all types of foreign DNA (Ablasser and Chen, 2019; Chen et al., 2016; Wu et al., 2021). However, how Ox-mtDNA reaches the cytoplasm to bind NLRP3 and whether NRLP3 activators also activate cGAS-STING signaling was heretofore unknown. In fact, it was reported that stressed mitochondria release their entire DNA content to activate cGAS-STING signaling with no need for extracellular stimuli (Kim et al., 2019; West et al., 2015; West and Shadel, 2017; Wu et al., 2021) and that activated Casp1 cleaves cGAS and dampens cGAS-STING-mediated IFN production (Wang et al., 2017). Applicant now show that Ox-mtDNA generation precedes NLRP3 inflammasome assembly and explain how Ox-mtDNA escapes mitochondria to license NLRP3 inflammasome as well as cGAS-STING activation. Applicant's results demonstrate that mtDNA oxidation has two distinct functions, as it triggers mtDNA fragmentation and generates a specific NLRP3 ligand (Shimada et al., 2012; Zhong et al., 2018). Indeed, the cytosolic pool of mtDNA consists of sub-genomic fragments whose generation was heretofore not understood and the role of mitochondrial BER in regulating the crosstalk between the NLRP3 inflammasome and cGAS-STING pathways was enigmatic. Applicant's studies solve these puzzles and chart a unified pathway through which NLRP3 activators generate Ox-mtDNA, which gets fragmented, released into the cytosol, and induces NLRP3 inflammasome assembly and STING phosphorylation. The earliest molecular event in this pathway is rapid MCU-dependent [Ca²⁺]m uptake, occurring within 30-60 sec. post-stimulation. [Ca²⁺]m triggers mPTP opening in the IMM to induce VDAC oligomerization in the OMM. Concurrently, NLRP3 activators trigger mtROS production, which is further enhanced by mitochondrial depolarization due to mPTP and VDAC opening and leads to oxidation of mtDNA, a prerequisite for its fragmentation. Ox-mtDNA generated via this pathway is either repaired by the BER enzyme OGG1, which attenuates NLRP3 inflammasome and cGAS-STING activation, or is cleaved by FEN1 to generate small DNA fragments (<650 bp) that are released to the cytoplasm via mPTP-dependent VDAC channels. These findings are consistent with a previous study showing that inclusion of VDAC1 in liposomes increases mtDNA passage across the lipid membrane and suggesting that the mtDNA region that is preferentially released is the D-loop region (Kim et al., 2019). In the cytoplasm, Ox-mtDNA fragments interact with NLRP3 to trigger inflammasome assembly and Casp1 activation, and also lead to STING phosphorylation at Ser³⁶⁵, which is required for IRF3 binding and activation of the IFN response (Chen et al., 2016; Liu et al., 2015; Tanaka and Chen, 2012). Exactly how FEN1, which removes flap structures and RNA primers generated during DNA replication and repair and collaborates with the degradosome to drive apoptotic DNA fragmentation (Parrish et al., 2003; Zheng et al., 2011), cleaves Ox-mtDNA is not clear. It is plausible that FEN1 acts on unique structures generated by DNA oxidation or during Ox-mtDNA repair, suggesting that release of mtDNA fragments from stressed mitochondria requires oxidation even when no obvious external stimuli are implicated. Interference with any of the critical steps described above attenuates IL-1β and ccf-mtDNA release, as well as STING phosphorylation, providing additional targets for anti-inflammatory drugs that can inhibit all three pathways.

Applicant's results also show that VDAC oligomerization is a signal-responsive event that depends on mPTP opening, which contrary to previous reports (Bernardi et al., 2006; Perez-Trevino et al., 2020), is not affected by mtDNA oxidation per se. Oxidative stress and mtDNA were also suggested to trigger VDAC oligomerization (Kim et al., 2019). However, Applicant find that mtROS do not induce VDAC oligomerization, which is not affected by mtDNA depletion, supporting the notion that poorly-defined mPTP components (Karch and Molkentin, 2014) rather than Ox-mtDNA fragments, trigger VDAC oligomerization. Without being bound by theory, these results also explain the origin of ccf-mtDNA in numerous inflammatory conditions including AD, NASH, heart failure, atherosclerosis, diabetes, lupus, and rheumatoid arteritis (Duvvuri and Lood, 2019; Zhong et al., 2019). Both mt-OGG1 expression and FEN1 inhibition substantially suppressed ccf-mtDNA appearance in BALF and Ox-mtDNA in peritoneal exudates. Once in the cytoplasm, Ox-mtDNA is secreted along with IL-1β and IL-18 via Gasdermin D pores and subsequently up-taken by bystander cells via clathrin-mediated endocytosis to activate cGAS-STING. Although active Casp1 cleaves cGAS and blocks the response to viral DNA infection (Wang et al., 2017), the balance between NLRP3 inflammasome activation and cGAS-STING signaling is likely to be cell type dependent and modulated by NLRP3 expression. As demonstrated above, cells that express low NLRP3 amounts undergo more robust cGAS-STING activation, whose final outcome in LPS-primed macrophages is overshadowed by the TLR4 induced IFN response (Liu et al., 2015). Nonetheless, the pathway of Ox-mtDNA fragmentation and cytosolic release described above can result in production of three different classes of systemic inflammatory mediators by suitably activated myeloid cells: IL-1β and IL-18, IFNs and ccf-mtDNA.

### Experimental Model and Subject Details

### Animal Studies

8-week-old male C57BL/6 mice were purchased from Charles River Laboratories or bred at UCSD. mt-Ogg1Tg mice in the C57BL/6 background (Wang et al., 2011) were kindly provided by Dr. Lyudmila Rachek, University of South Alabama and Dr. Lars Eide, University of Oslo and Oslo University Hospital. Nlrp3−/−mice in the C57BL/6 background were kindly provided by Dr. Hal M. Hoffman (UCSD). All mice were bred and maintained at UCSD and handled in accordance with Institutional Animal Care and Use Committee and NIH guidelines.

### LPS-Induced ARDS

8-12-week-old male WT and mt-Ogg1Tg mice were allocated randomly and subjected to LPS-induced ARDS as described (Xian et al., 2021). Briefly, 5 mg/kg LPS or vehicle was i.p. injected and the mice were euthanized 24 h later. At the experimental endpoint, the lungs were inflated with cold PBS, excised and fixed in 10% formalin for histological evaluation.

### Alum-Induced Peritonitis

8-12-week-old male C57BL/6 mice and WT or mt-Ogg1Tg mice were allocated randomly and i.p. injected with alum (1 mg dissolved in 0.2 mL sterile PBS) or PBS. Peritoneal lavage was collected 4 h post-injection to measure cytokine amounts by ELISA. Another batch of mice allocated randomly were i.p. injected with alum (300 ug) or PBS and after 16 h mice were euthanized, and peritoneal cavities were washed with 6 mL cold sterile PBS. Neutrophils (CD11b⁺Ly6G⁺F4/80⁻) and monocytes (CD11b⁺Ly6C⁺Ly6G⁻) present in peritoneal lavage fluid were quantified by flow cytometry. For blocking Fc-mediated interactions, mouse cells were pre-incubated with 0.5-1 μg of purified anti-mouse CD16/CD32 per 100 μL. Isolated cells were stained with labelled antibodies in PBS with 2% FCS and 2 mM EDTA or cell staining buffer (Biolegend). Dead cells were excluded based on staining with propidium iodide (PI). Absolute numbers of immune cell subtypes in the peritoneum were calculated by multiplying total peritoneal cell numbers by percentages of immune cell subtypes amongst total cells. Cells were analyzed on a Beckman Coulter Cyan ADP flow cytometer.

To measure ccf-mtDNA content in BALF or peritoneal cavity, the collected BALF or peritoneal lavage was centrifuged for 10 minutes at 1200 rpm to remove cells and cellular debris. DNA were isolated from 400 μL of the supernatant using the QIAmp 96 DNA Blood Kit according to the manufacturer's instruction for blood and body-fluid protocol. The isolated DNA was then eluted in 300 μL and quantified using spectrophotometric analysis at 260/280 nm with Nanodrop and analyzed by qPCR.

### Cell Lines and Primary Culture

### Macrophage Culture and Stimulation

Femurs and tibias from C57BL/6, mt-Ogg1^{Tg} (bred at UCSD) and Ogg1^{-/-} (provided by Dr. Harini Sampath) mice above 8 weeks of age (regardless of gender) were used to generate bone-marrow-derived macrophages (BMDM). Bone marrow cells were cultured in high glucose DMEM supplemented with 10% FBS, 20% L929-cell conditioned medium, and 100 U/mL penicillin-streptomycin for 7-10 days at 37° C. with 5% CO2 (Hornung et al., 2008). NLRP3 inflammasome activation was induced after 4 h priming with ultrapure LPS (200 ng/ml) by challenge with the NLRP3 activators ATP (4 mM) or nigericin (10 μM) for 1 h, and monosodium ureate (MSU) crystals (600 μg/mL) or alum (500 μg/mL) for 6 h. XeC (5 μM), RuR (10 μM), CsA (1 μM), VBIT-4 (10 μM), FEN1-IN-4 (10 μM unless otherwise indicated), DMNQ (20 μM) were added 16 h, while MitoParaquat (5 μM) was added 1 h, before LPS or NLRP3 activators challenge. Etoposide was used at 10 μg/mL for 16 h to induce apoptosis in BMDM.

Supernatants and cell lysates were collected for ELISA and immunoblot (IB) analyses.

### Method Details

### shRNA Lentiviral Knockdown

Gene silencing was performed by lentiviral transduction of primary BMDM as described (Xian et al., 2021). Sequences of specific shRNAs used in this study were obtained from the MISSION shRNA Library (Sigma). Lentiviral particles generated using VSV-G, PLV-CMVΔ8.9 plasmids and specific shRNAs in HEK293T cells were used to knockdown Ogg1, Mgme1, Mre11a, Fen1, Vdac1 or Vdac3. Supernatants were collected 48 h after transfection, filtrated through a 0.45 micron pore filter and added to primary BMDM. To increase infection efficiency, 8 μg/mL of polybrene were added. The virus containing medium was washed after 6 h and the cells were cultured with fresh medium. Infected cells were expanded and selected with puromycin at 72 h post-transduction.

### Protein Immunoblotting

Whole cell lysates were prepared in RIPA buffer (25 mM Tris-HCl pH 7.6, 150 mM NaCl, 1% NP-40, 1% sodium deoxycholate, 0.1% SDS) containing a protease inhibitor cocktail and a phosphatase inhibitor cocktail. Protein concentrations were determined using BCA Protein Assay Kit. Equal amounts of protein were separated by SDS-PAGE and transferred onto PVDF membranes, blocked in 5% BSA in 1×TBST for 1 h and incubated with primary antibodies overnight. Secondary antibodies were added for 1 h and detection was performed using Clarity Western ECL Substrate.

### ELISA (Enzyme-Linked Immunosorbent assay)

Paired antibodies (capture and detection) and standard recombinant mouse IL-1β and TNF were used to determine mouse cytokine concentrations according to manufacturer's instructions.

### Immunofluorescence and Confocal Microscopy

BMDM were fixed in 4% paraformaldehyde (PFA), permeabilized in 0.2% Triton X-100,and blocked in 1×PBS supplemented with 3% BSA. Primary antibodies were incubated in blocking buffer at 4° C. overnight. Secondary Alexa antibodies were added for 1 h. Nuclei were counterstained with DAPI. Samples were imaged through a Leica SP5 confocal microscope.

[Ca²⁺]m was measured in BMDM loaded with 5 μM Rhod-2, AM and 10 nM MitoTracker™ Green for 30 minutes at 37° C. with 5% CO2. After washing, BMDM were kept in the incubator for additional 30 minutes before confocal imaging (Nikon CSU-X1 Spinning Disk). Dynamic [Ca²⁺]m images were captured at 10 seconds intervals for 4 minutes, with the addition of NLRP3 agonist at 40 seconds.

### Histological Evaluation and Immunohistochemistry

Lungs were fixed in 10% formalin for 24 h and embedded in paraffin, sectioned and stained with hematoxylin and eosin (H&E) to evaluate gross morphology and lung damage and inflammation, and with Sirius red to determine collagen buildup. Lung sections were incubated with antibodies specific for F4/80 (macrophage marker) and myeloperoxidase (MPO, neutrophil marker). Stained areas were quantified with ImageJ software.

### RNA Isolation and Quantitative Real-Time PCR (qPCR)

RNA was extracted using AllPrep DNA/RNA Mini kit, and cDNA was synthesized using SuperScript™ VILO™ cDNA Synthesis Kit. mRNA expression was determined by QPCR in a CFX96 thermal cycler (Biorad) as described (Xian et al., 2021). Data are presented in arbitrary units and calculated by the 2\hat{ }(−ΔΔCT) method. Primer sequences were obtained from the NIH qPrimerDepot (http://mouseprimerdepot.nci.nih.gov) and provided by Integrated DNA technologies.

### Mitochondrial Function

Mitochondrial membrane potential was measured using TMRM according to manufacturer's instructions. Briefly, BMDM were primed or not with LPS for 4 h, and further stimulated with ATP or nigericin for 60 minutes. Cells were incubated with 200 nM TMRM for 30 minutes at 37° C. After washing twice, fluorescence intensity was determined per manufacturer's instructions using a FilterMax F5 multimode plate reader (Molecular Devices). Mitochondrial reactive oxygen species (mtROS) were measured using MitoSOX (Invitrogen) as described (Xian et al., 2021). BMDM treated as indicated were loaded with 4 μM MitoSOX for 20 minutes. After washing with PBS twice, cells were resuspended in PBS and counted. Equal numbers of cells from different treatment groups were then plated onto 96-well plates for fluorescence reading. To quantify [Ca²⁺]m by 5 μM Rhod-2 or intracellular Ca²⁺ intensity by 1 μM Fluo-4, BMDM treated as indicated were loaded with the respective dyes according to manufacturer's instructions for 30 minutes. After washing twice, fluorescence intensity was determined using a FilterMax F5 multimode plate reader (Molecular Devices). To analyze mPTP opening by calcein-quenching assay, BMDM treated as indicated were loaded with calcein at 2 μM and CoCl2 at 160 μM for 30 minutes at 37° C. according to manufacturer's instructions. After washing twice, fluorescence intensity was determined per with a FilterMax F5 multimode plate reader (Molecular Devices).

### Measurement of Total mtDNA

Macrophages were primed with LPS for 4 h and total DNA was isolated using AllPrep DNA/RNA Mini Kit according to manufacturer's instructions. mtDNA was quantified by qPCR using primers specific for the mitochondrial D-loop region, cytochrome c oxidase (Cox1) or a specific region of mtDNA that is not inserted into nuclear DNA (non-NUMT). Nuclear DNA encoding telomerase reverse transcriptase (Tert), 18S ribosomal RNA and β2 microglobulin (B2m) was used for normalization.

### Cellular Fractionation and Measurement of Cytosolic mtDNA

BMDM were stimulated as indicated. Cellular fractionation was carried out as previously described (Xian and Liou, 2019; Xian et al., 2019). Briefly, BMDM were washed with PBS and harvested, 10% of which were saved for DNA extraction of whole cells. The remaining BMDM resuspended with pre-chilled mitochondrial extraction buffer 1 (220 mM mannitol, 70 mM sucrose, 20 mM HEPES-KOH, pH 7.5, 1 mM EDTA and 2 mg/mL bovine serum albumin) supplemented with a protease inhibitor cocktail and a phosphatase inhibitor cocktail were passed through a 25-G syringe (BD Biosciences) 20 times on ice. The homogenized cells were centrifuged at 1000×g for 15 minutes at 4° C. The supernatant was further centrifuged at 10,000×g for 10 minutes at 4° C. to pellet the mitochondria from supernatant cytosolic fraction. qPCR was performed after DNA purification with AllPrep DNA/RNA Mini Kit according to manufacturer's instructions, from both whole cell extracts and cytosolic fractions using mtDNA primers (D-loop, Cox1, non-NUMT), or after DNA purification from whole cell extracts using nuclear DNA primers (Tert, 18S, B2m). The Ct values obtained for mtDNA abundance in whole cell extracts served as normalization controls for the mtDNA values obtained from the cytosolic fractions. For the measurement of Ox-mtDNA, purified mtDNA was extracted from the cytosolic or mitochondrial fractions as indicated. The 8-OH-dG content was then quantified using 8-hydroxy 2-deoxyguanosine ELISA Kit, per manufacturer's instructions.

### Preparation of Extracellular mtDNA and Stimulation of Naïve Macrophages by Cell-Free mtDNA

Extracellular DNA of donor macrophages (1500,000 cells per group) was isolated using QIAamp Circulating Nucleic Acid Kit according to manufacturer's instructions and then eluted in 300 μL distilled H2O and quantified using spectrophotometric analysis at 260/280 nm with Nanodrop. DNA amounts purified among the different treatment groups vary. The DNA amount secreted from LPS-primed donor BMDM is low, approximately at 0.1 μg. The amount of DNA released by LPS-primed donor BMDM challenged with ATP is about 1.3 μg, while the amount of DNA released by LPS-primed donor BMDM challenged with nigericin is around 2 μg. The amount of DNA secreted by LPS-primed donor BMDM challenged with MSU or alum is about 0.7 μg. Applicant used 5% of the prepared DNA for qPCR measurement of extracellular mtDNA and the remaining 95% were added to the recipient BMDM for analyzing the paracrine immunostimulatory effects.

### PCR

DNA was isolated using AllPrep DNA/RNA Mini Kit according to manufacturer's instructions and amplified using Phusion Green Hot Start II High-Fidelity PCR Master Mix, containing 100 nM of each primer.

16299 bp PCR was carried out using 200 ng DNA extracted from mitochondria as the template. 5698 bp PCR was carried out using 5 ng DNA extracted from mitochondria or 50-200 ng DNA extracted from cytoplasm as the template. D-loop 591 bp PCR was performed using 5 ng DNA as the template. The following PCR conditions were used. Hot start at 98° C. for 3 minutes, melting temperature of 98° C. for 10 seconds, annealing temperature of 60° C. (for 5698 bp and 591 bp) or 53° C. (for 16299 bp) for 30 seconds and extension temperature of 72° C. for 30 seconds (591 bp), 3 minutes (5698 bp) or 8 minutes 30 seconds (16299 bp), with 20 (591 bp), 35 (5698 bp) or 40 (16299 bp) cycles, followed by 72° C. for 20 minutes and kept at 4°. Amplified products were analyzed on 0.8%-1.2% agarose gels stained with ethidium bromide.

To analyze DNA fragmentation and extrusion in mitochondrial and cytosolic fractions, BMDM (15000,000 cells per group) were stimulated as indicated and fractionalized as above. To extract the nuclear fractions, BMDM lysed in 400 μL buffer 2 (50 mM HEPES, 150 mM NaCl, and 100 μg/mL digitonin) were rotated at 4° C. for 10 minutes. Cell homogenates were centrifuged at 4000×g for 2 minutes at 4° C. and pellets were washed twice with cold PBS and obtained as nuclear fraction. Cytosolic, mitochondrial or nuclear DNAs were purified using AllPrep DNA/RNA Mini Kit according to manufacturer's instructions and analyzed on 1% agarose gels stained with ethidium bromide.

To determine mtDNA oxidative damage using Fpg-sensitive qPCR analysis (Fpg removes oxidized purines from DNA and creates single-strand breaks, therefore blocks PCR amplification at these sites. Differences in qPCR cycles between Fpg-treated and untreated DNA are thereby a specific indicator of the presence of oxidative base damage), one aliquot of purified ccf-DNA (250 ng) was incubated with 8 units of Fpg in 1×NEBuffer 1 and 100 μg/mL BSA in a volume of 50 μL at 37° C. for 1 h. Fpg was then inactivated by heating at 60° C. for 5 minutes. 10 ng DNA was subsequently used for the qPCR assay to detect Fpg-sensitive cleavage sites. Data are presented as the ratio of Fpg-insensitive DNA, calculated as the quotient of signal intensities in Fpg-treated relative to untreated DNA (Pastukh et al., 2016).

### VDAC Cross-Linking Assay

BMDM treated as indicated were washed and collected in PBS, incubated for 40 min at 30° C. with the cross-linking reagent EGS (200 μM). Samples were subjected to SDS-PAGE and IB analyzed with anti-VDAC antibody.

### Quantification and Statistical Analysis

Data are shown as mean±s.d. Statistical significance was determined using two-tailed unpaired Student's t test. P values lower than 0.05 were considered statistically significant.

### Equivalents

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

TABLE 2
Key resources table

| | | |
|---|---|---|
| REAGENT or RESOURCE | SOURCE | IDENTIFIER |

Antibodies

| | | |
|---|---|---|
| IL-1β | Cell Signaling | Cat#12426; |
| | | N/A |
| ASC | Cell Signaling | Cat#67824; |
| | | N/A |
| VDAC | Cell Signaling | Cat#4161; |
| | | RRID: AB_10557420 |
| Tom20 | Cell Signaling | Cat#42406; |
| | | RRID: AB_2687663 |
| COX1/MT-CO1 | Cell Signaling | Cat#62101; |
| | | N/A |
| Phospho-STING (Ser365) | Cell Signaling | Cat#62912; |
| | | RRID: AB_2799635 |
| cGAS | Cell Signaling | Cat#31659; |
| | | RRID: |
| | | AB_2799008 |
| Cleaved Caspase-9 (Asp353) | Cell Signaling | Cat#9509; |
| | | RRID: AB_ |
| | | 2073476 |
| Cleaved Caspase-3 (Asp175) | Cell Signaling | Cat#9661; |
| | | RRID: AB_ |
| | | 2341188 |
| Cytochrome c (6H2.B4) | Cell Signaling | Cat#12963; |
| | | RRID: AB_ |
| | | 2637072 |
| Brilliant violet 510™ anti-mouse CD45 | BioLegend | Cat#103137; |
| | | RRID: AB_2561392 |
| Anti-rabbit IgG, HRP-linked Antibody | Cell Signaling | Cat#7074; |
| | | RRID: AB_2099233 |
| Anti-mouse IgG, HRP-linked Antibody | Cell Signaling | Cat#7076; |
| | | RRID: AB_330924 |
| MGME1 | Proteintech | Cat#23178-1-AP; |
| | | RRID: AB_2879223 |
| OGG1 | Santa Cruz | Cat#376935; |
| | Biotechnology | N/A |
| FEN1 | Santa Cruz | Cat#28355; |
| | Biotechnology | RRID: AB_627587 |
| MRE11A | Santa Cruz | Cat#135992; |
| | Biotechnology | RRID: AB_2145244 |
| TIM23 | Santa Cruz | Cat#514463; |
| | Biotechnology | N/A |
| Tubulin | Sigma-Aldrich | Cat#T9026; N/A |
| Caspase-1 | Adipogen | Cat#AG-20B- |
| | | 0042-C100; N/A |
| ASC | Adipogen | Cat#AG-25b- |
| | | 0006-C100; N/A |
| NLRP3 | Adipogen | Cat#AG-20B- |
| | | 0014-C100; N/A |
| F4/80 | Invitrogen | Cat#MF48000; |
| | | RRID: AB_1500089 |
| Myeloperoxidase | Abcam | Cat#ab9535; |
| | | RRID: AB_307322 |
| Anti-ATP Synthase, beta chain, clone | Millipore | Cat#MAB3494; |
| 4.3E8.D1 antibody | | RRID: AB_177597 |
| Mouse IL-1beta/IL-1F2 Antibody | R&D Systems | Ca#MAB401; |
| | | RRID: AB_2124620 |
| Mouse IL-1beta/IL-1F2 Biotinylated | R&D Systems | Ca#BAF401; |
| Antibody | | RRID: |
| | | AB_356450 |
| Anti-Mouse/Rat TNF alpha antibody | eBioscience | Cat#14-7423; |
| | | RRID: AB_468492 |
| Anti-Mouse/Rat TNF alpha Biotin antibody | eBioscience | Cat#13-7341; |
| | | RRID: AB_466951 |
| FITC anti-mouse CD11b | eBioscience | Cat#11-0112-82; |
| | | RRID: AB_464935 |
| PE-Cy7 anti-mouse Gr-1 | eBioscience | Cat#25-5931-82; |
| | | RRID: AB_469663 |
| eFluor 450 anti-mouse Ly-6C | eBioscience | Cat#48-5932-80; |
| | | RRID: AB_10805518 |
| PE anti-mouse F4/80 | eBioscience | Cat#12-4801-82; |
| | | RRID: AB_465923 |
| CD16/CD32 | eBioscience | Cat#14-0161-86; |
| | | RRID: AB_467135 |
| Donkey anti-rabbit IgG (H + L) secondary | Invitrogen | Cat#A-21206; |
| antibody, Alexa Fluor 488 | | RRID: AB_2535792 |
| Donkey anti-rabbit IgG (H + L) secondary | Invitrogen | Cat#A-21207; |
| antibody, Alexa Fluor 594 | | RRID: AB_141637 |
| Donkey anti-mouse IgG (H + L) secondary | Invitrogen | Cat#A-21202; |
| antibody, Alexa Fluor 488 | | RRID: AB_141607 |
| Donkey anti-mouse IgG (H + L) secondary | Invitrogen | Cat#A-21203; |
| antibody, Alexa Fluor 594 | | RRID: AB_141633 |
| Donkey anti-rat IgG (H + L) secondary | Invitrogen | Cat#A-21209; |
| antibody, Alexa Fluor 594 | | RRID: AB_2535795 |

Chemicals, Peptides, and Recombinant Proteins

| | | |
|---|---|---|
| Recombinant Mouse IL-1β | R&D Systems | Cat#401-ML |
| Recombinant Mouse TNFα | eBioscience | Cat#14-8321 |
| Ultrapure LPS, E. coli 0111:B4 | Invivogen | Cat#tlrl-3pelps |
| Lipopolysaccharides from Escherichia | Sigma-Aldrich | Cat#L2654 |
| coli O26:B6 | | |
| Adenosine 5′-triphosphate disodium | Sigma-Aldrich | Cat#A6559 |
| salt solution | | |
| Cyclosporine A (CsA) | Sigma-Aldrich | Cat#C3662 |
| Phosphatase Inhibitor Cocktail 2 | Sigma-Aldrich | Cat#P5726 |
| Triton X-100 | Sigma-Aldrich | Cat#T8787 |
| Dimethyl Sulfoxide (DMSO) | Sigma-Aldrich | Cat#D2650-100 |
| Polybrene | Sigma-Aldrich | Cat#TR-1003 |
| Direct Red 80 | Sigma-Aldrich | Cat#365548 |
| Picric acid solution | Sigma-Aldrich | Cat#5860-16 |
| Ethidium bromide | Sigma-Aldrich | Cat#E7637 |
| FluorSave™ | Millipore | Cat#345789 |
| Ruthenium red (RuR) | EMD Millipore | Cat#557450 |
| FEN1-IN-4 | Selleckchem | Cat#S3397 |
| MitoParaquat | Selleckchem | Cat#S8978 |
| DMNQ | Selleckchem | Cat#S6563 |
| Xestospongin C (XeC) | Cayman | Cat#64950. |
| VBIT-4 | MedChemExpress | Cat#HY-129122 |
| Etoposide | MedChemExpress | Cat#HY-13629 |
| Pitstop2 | MedChemExpress | Cat#HY-115604 |
| Staurosporine | MedChemExpress | Cat#HY-15141 |
| Camptothecin | MedChemExpress | Cat# HY-16560 |
| Nigericin | Invivogen | Cat#tlrl-nig |
| Monosodium urate (MSU) | Enzo Life Science | Cat#ALX-400- |
| | | 047-M002 |
| Imject™ Alum Adjuvant | Thermo Scientific | Cat#77161 |
| Ethylene glycol bis (succinimidyl | Thermo Scientific | Cat#21565 |
| succinate) (EGS) | | |
| Phusion Green Hot Start II High-Fidelity | Thermo Scientific | Cat#F566S |
| PCR Master Mix | | |
| DNase I | Thermo Scientific™ | EN0521 |
| DAPI (4′6-Diamidino-2-Phenylindole, | Invitrogen | Cat#D1306 |
| Dihydrochloride) | | |
| Tetramethylrhodamine, Methyl Ester, | Invitrogen | Cat#T668 |
| Perchlorate (TMRM) | | |
| MitoSOX™ Red mitochondrial superoxide | Invitrogen | Cat#M36008 |
| indicator | | |
| Rhod-2, AM | Invitrogen | Cat#R1244 |
| Fluo-4, AM | Invitrogen | Cat#F14201 |
| MitoTracker™ Green FM | Invitrogen | Cat#M7514. |
| Complete™, Mini Protease Inhibitor | Roche | Cat#11836153001 |
| Cocktail | | |
| EDTA | Versene solution | Cat#9314 |
| Clarity Western ECL Substrate | Biorad | Cat#1705061 |
| Bovine Serum Albumin | Gemini | Cat#700-100 |
| Protein Assay Dye reagent concentrate | Biorad | Cat#5000006 |
| Hematoxyline 560 | Leica | Cat#3801570 |
| Alcoholic Eosine Y 515 | Leica | Cat#3801615 |
| Picric acid solution | Ricca | Cat#5860-16 |
| Fpg enzyme | New England | Cat#M0240S |
| | BioLabs Inc. | |

Critical Commercial Assays

| | | |
|---|---|---|
| MitoProbe™ Transition Pore Assay Kit | Invitrogen | Cat#M34153. |
| AllPrep DNA/RNA Mini kit | Qiagen | Cat#80204 |
| QIAamp DNA Blood Mini Kit | Qiagen | Cat#51106 |
| RNeasy Plus Mini kit | Qiagen | Cat#74134 |
| QIAamp Circulating Nucleic Acid Kit | Qiagen | Cat#55114 |
| SuperScript™ VILO™ CDNA Synthesis Kit | Invitrogen | Cat#11754050 |
| SsoAdvanced Universal SYBR Green | Biorad | Cat#172-5274 |
| Supermix | | |
| Power SYBR® Green RNA-to-Ct™ 1-Step Kit | Applied Biosystems | Cat#4391178 |
| Lipofectamine™ 3000 Transfection Reagent | Invitrogen | Cat#L3000001 |
| LDH-Glo™ Cytotoxicity Assay | Promega | Cat#J2380 |
| 8-hydroxy 2 deoxyguanosine ELISA Kit | Abcam | Cat#ab201734 |

Experimental Models: Cell Lines

| | | |
|---|---|---|
| HEK293T | ATCC | ATCC® CRL- |
| | | 3216™; |
| | | RRID: CVCL_0063 |
| L929 | ATCC | ATCC® CCL-1™; |
| | | RRID: CVCL_0462 |

Experimental Models: Organisms/Strains

| | | |
|---|---|---|
| Mouse: C57BL/6N ( ) | In house breeding | N/A |
| Mouse: C57BL/6N (in vivo) | Charles River | N/A |
| | Laboratories | |
| Mouse: mt-Ogg1^{Tg} | In house breeding | N/A |
| Mouse: Nlrp3^{-/-} | In house breeding | N/A |
| Mouse: Gsdmd^{-/-} | Dr. Hal M. Hoffman | N/A |
| Mouse: cGAS^{-/-} | Dr. Wei Ying | N/A |
| Mouse: Sting^{-/-} | Dr. Dennis Carson | N/A |
| Mouse: Ogg1^{-/-} | Dr. Harini Sampath | N/A |

Oligonucleotides

| | | |
|---|---|---|
| Primers for mouse D-loop | Integrated DNA | N/A |
| Forward: AATCTACCATCCTCCGTGAAACC | Technologies | |
| (SEQ ID NO: 1) | | |
| Reverse: TCAGTTTAGCTACCCCCAAGTTTAA | | |
| (SEQ ID NO: 2) | | |
| Primers for mouse 18S | Integrated DNA | N/A |
| Forward: TAGAGGGACAAGTGGCGTTC (SEQ | Technologies | |
| ID NO: 3) | | |
| Reverse: CGCTGAGCCAGTCAGTGT (SEQ ID | | |
| NO: 4) | | |
| Primers for mouse Tert | Integrated DNA | N/A |
| Forward: CTAGCTCATGTGTCAAGACCCTCTT | Technologies | |
| (SEQ ID NO: 5) | | |
| Reverse: GCCAGCACGTTTCTCTCGTT (SEQ ID | | |
| NO: 6) | | |
| Primers for mouse Cox1 | Integrated DNA | N/A |
| Forward: GCCCCAGATATAGCATTCCC (SEQ | Technologies | |
| ID NO: 7) | | |
| Reverse: GTTCATCCTGTTCCTGCTCC (SEQ ID | | |
| NO: 8) | | |
| Primers for mouse Non-Numt | Integrated DNA | N/A |
| Forward: CTAGAAACCCCGAAACCAAA (SEQ | Technologies | |
| ID NO: 9) | | |
| Reverse: CCAGCTATCACCAAGCTCGT (SEQ | | |
| ID NO: 10) | | |
| Primers for mouse B2m | Integrated DNA | N/A |
| Forward: ATGGGAAGCCGAACATACTG (SEQ | Technologies | |
| ID NO: 11) | | |
| Reverse: CAGTCTCAGTGGG GGTGAAT (SEQ | | |
| ID NO: 12) | | |
| Primers for mouse Hprt1 | Integrated DNA | N/A |
| Forward: CTGGTGAAAAGGACCTCTCG (SEQ | Technologies | |
| ID NO: 13) | | |
| Reverse: | | |
| TGAAGTACTCATTATAGTCAAGGGCA (SEQ | | |
| ID NO: 14) | | |
| Primers for mouse Il1b | Integrated DNA | N/A |
| Forward: AGTTGACGGACCCCAAAAG (SEQ | Technologies | |
| ID NO: 15) | | |
| Reverse: AGCTGGATGCTCTCATCAGG (SEQ | | |
| ID NO: 16) | | |
| Primers for mouse Il6 | Integrated DNA | N/A |
| Forward: CCAGGTAGCTATGGTACTCCA (SEQ | Technologies | |
| ID NO: 17) | | |
| Reverse: GCTACCAAACTGGCTATAATC (SEQ | | |
| ID NO: 18) | | |
| Primers for mouse Tnf | Integrated DNA | N/A |
| Forward: CCCTCACACTCAGATCATCTT (SEQ | Technologies | |
| ID NO: 19) | | |
| Reverse: GCTACGACGTGGGCTACAG (SEQ ID | | |
| NO: 20) | | |
| Primers for mouse Cxcl10 | Integrated DNA | N/A |
| Forward: ATCATCCCTGCGAGCCTATCCT | Technologies | |
| (SEQ ID NO: 21) | | |
| Reverse: GACCTTTTTTGGCTAAACGCTTTC | | |
| (SEQ ID NO: 22) | | |
| Primers for mouse Isg15 | Integrated DNA | N/A |
| Forward: CATCCTGGTGAGGAACGAAAGG | Technologies | |
| (SEQ ID NO: 23) | | |
| Reverse: CTCAGCCAGAACTGGTCTTCGT | | |
| (SEQ ID NO: 24) | | |
| Primers for mouse Ifit3 | Integrated DNA | N/A |
| Forward: GCTCAGGCTTACGTTGACAAGG | Technologies | |
| (SEQ ID NO: 25) | | |
| Reverse: CTTTAGGCGTGTCCATCCTTCC (SEQ | | |
| ID NO: 26) | | |
| Primers for mouse Irf7 | Integrated DNA | N/A |
| Forward: CCTCTGCTTTCTAGTGATGCCG | Technologies | |
| (SEQ ID NO: 27) | | |
| Reverse: CGTAAACACGGTCTTGCTCCTG | | |
| (SEQ ID NO: 28) | | |
| Primers for mouse D-loop 591 bp | Integrated DNA | N/A |
| Forward: GTGTTATCTGACATACACCATACAG | Technologies | |
| (SEQ ID NO: 29) | | |
| Reverse: TGGGAACTACTAGAATTGATCAGGA | | |
| (SEQ ID NO: 30) | | |
| Primers for mouse mt-DNA 5698 bp | Integrated DNA | N/A |
| Forward: ATGATTTATCTCAACCCTAGCAG | Technologies | |
| (SEQ ID NO: 31) | | |
| Reverse: GTAGTGGGACTTCTAGAGGGTTAAGT | | |
| (SEQ ID NO: 32) | | |
| Primers for mouse mt-DNA 16299 bp | Integrated DNA | N/A |
| Forward: ACTGAAAATGCTTAGATGGATAATTGTA | Technologies | |
| (SEQ ID NO: 33) | | |
| Reverse: GTATGATTAGAGTTTTGGTTCACGGAACA | | |
| (SEQ ID NO: 34) | | |

Recombinant DNA

| | | |
|---|---|---|
| VSV-G | (Zhong et al., 2016c) | N/A |
| pLV-CMVΔ8.9 | (Zhong et al., 2016c) | N/A |
| shOgg1 #1: 5′- | Sigma shRNA | N/AN/A |
| CCGGCAAGTCAGAAAGACTTAACATCTCGA | Mission | |
| GATGTTAAGTCTTTCTGACTTGTTTTTG-3′ | librarySigma shRNA | |
| (SEQ ID NO: 35) | Mission library | |
| shOgg1 #2: | Sigma shRNA | N/A |
| 5′-CCGGGATGTCCATGTATGGCAGATTCTCGA | Mission library | |
| GAATCTGCCATACATGGACATCTTTTTG-3′ | | |
| (SEQ ID NO: 36) | | |
| shMgme1: | Sigma shRNA | N/A |
| 5′-CCGGGCCCAGTCTTTGAATTGAGATCTCGA | Mission library | |
| GATCTCAATTCAAAGACTGGGCTTTTTTG-3′ | | |
| (SEQ ID NO: 37) | | |
| shMre11a: | Sigma shRNA | N/A |
| 5′-CCGGCGCCATATTGATGCTCTGGAACTCGA | Mission library | |
| GTTCCAGAGCATCAATATGGCGTTTTTG-3′ | | |
| (SEQ ID NO: 38) | | |
| shFen1: | Sigma shRNA | N/A |
| 5′-CCGGGAATGACATCAAGAGCTACTTCTCGA | Mission library | |
| GAAGTAGCTCTTGATGTCATTCTTTTTG-3′ | | |
| (SEQ ID NO: 39) | | |
| shVdac1: | Sigma shRNA | N/A |
| 5′-CCGGGAGTTGATAAATACCACGTTACTCGA | Mission library | |
| GTAACGTGGTATTTATCAACTCTTTTTG-3′ | | |
| (SEQ ID NO: 40) | | |
| shVdac3: | Sigma shRNA | N/A |
| 5′-CCGGTGACTCTTGATACCATATTTGCTCGAG | Mission library | |
| CAAATATGGTATCAAGAGTCATTTTTG-3′ | | |
| (SEQ ID NO: 41) | | |

Software and Algorithms

| | | |
|---|---|---|
| Prism 8.0 | GraphPad Software | N/A |
| Image J | Image J | N/A |

Other

| | | |
|---|---|---|
| DMEM | Gibco | Cat#11995-065 |
| Fetal Bovine Serum (FBS) | Gibco | Cat#10437-028 |
| | | (lot. 1913181) |
| Penicillin-streptomycin | Gibco | Cat#15140-122 |

### REFERENCES:

1. Ablasser, A., and Chen, Z.J. (2019). cGAS in action: Expanding roles in immunity and inflammation. Science 363.
2. Andreeva, L., David, L., Rawson, S., Shen, C., Pasricha, T., Pelegrin, P., and Wu, H. (2021). NLRP3 cages revealed by full-length mouse NLRP3 structure control pathway activation. Cell 184, 6299-6312 e6222.
3. Barber, G.N. (2015). STING: infection, inflammation and cancer. Nat Rev Immunol 15, 760-770.
4. Baughman, J.M., Perocchi, F., Girgis, H.S., Plovanich, M., Belcher-Timme, C.A., Sancak, Y., Bao, X.R., Strittmatter, L., Goldberger, O., Bogorad, R.L., et al. (2011). Integrative genomics identifies MCU as an essential component of the mitochondrial calcium uniporter. Nature 476, 341-345.
5. Bernardi, P., Krauskopf, A., Basso, E., Petronilli, V., Blachly-Dyson, E., Di Lisa, F., and Forte, M.A. (2006). The mitochondrial permeability transition from in vitro artifact to disease target. FEBS J 273, 2077-2099.
6. Broekemeier, K.M., Dempsey, M.E., and Pfeiffer, D.R. (1989). Cyclosporin A is a potent inhibitor of the inner membrane permeability transition in liver mitochondria. J Biol Chem 264, 7826-7830.
7. Broz, P., and Dixit, V.M. (2016). Inflammasomes: mechanism of assembly, regulation and signalling. Nat Rev Immunol 16, 407-420.
8. Chen, Q., Sun, L., and Chen, Z.J. (2016). Regulation and function of the cGAS-STING pathway of cytosolic DNA sensing. Nat Immunol 17, 1142-1149.
9. Croteau, D.L., and Bohr, V.A. (1997). Repair of oxidative damage to nuclear and mitochondrial DNA in mammalian cells. J Biol Chem 272, 25409-25412.
10. de Souza-Pinto, N.C., Hogue, B.A., and Bohr, V.A. (2001). DNA repair and aging in mouse liver: 8-oxodG glycosylase activity increase in mitochondrial but not in nuclear extracts. Free Radic Biol Med 30, 916-923.
11. Desler, C., Lillenes, M.S., Tonjum, T., and Rasmussen, L.J. (2018). The Role of Mitochondrial Dysfunction in the Progression of Alzheimer's Disease. Curr Med Chem 25, 5578-5587.
12. Dick, M.S., Sborgi, L., Ruhl, S., Hiller, S., and Broz, P. (2016). ASC filament formation serves as a signal amplification mechanism for inflammasomes. Nat Commun 7, 11929.
13. Duvvuri, B., and Lood, C. (2019). Cell-Free DNA as a Biomarker in Autoimmune Rheumatic Diseases. Front Immunol 10, 502.
14. Elliott, E.I., and Sutterwala, F.S. (2015). Initiation and perpetuation of NLRP3 inflammasome activation and assembly. Immunol Rev 265, 35-52.
15. Exell, J.C., Thompson, M.J., Finger, L.D., Shaw, S.J., Debreczeni, J., Ward, T.A., McWhirter, C., Sioberg, C.L., Martinez Molina, D., Abbott, W.M., et al. (2016). Cellularly active N-hydroxyurea FEN1 inhibitors block substrate entry to the active site. Nat Chem Biol 12, 815-821.
16. Fontana, G.A., and Gahlon, H.L. (2020). Mechanisms of replication and repair in mitochondrial DNA deletion formation. Nucleic Acids Res 48, 11244-11258.
17. Garcia, N., and Chavez, E. (2007). Mitochondrial DNA fragments released through the permeability transition pore correspond to specific gene size. Life Sci 81, 1160-1166.
18. Grailer, J.J., Canning, B.A., Kalbitz, M., Haggadone, M.D., Dhond, R.M., Andjelkovic, A.V., Zetoune, F.S., and Ward, P.A. (2014). Critical role for the NLRP3 inflammasome during acute lung injury. J Immunol 192, 5974-5983.
19. Gross, O., Thomas, C.J., Guarda, G., and Tschopp, J. (2011). The inflammasome: an integrated view. Immunol Rev 243, 136-151.
20. Gurung, P., Lukens, J.R., and Kanneganti, T.D. (2015). Mitochondria: diversity in the regulation of the NLRP3 inflammasome. Trends Mol Med 21, 193-201.
21. Halle, A., Hornung, V., Petzold, G.C., Stewart, C.R., Monks, B.G., Reinheckel, T., Fitzgerald, K.A., Latz, E., Moore, K.J., and Golenbock, D.T. (2008). The NALP3 inflammasome is involved in the innate immune response to amyloid-beta. Nat Immunol 9, 857-865.
22. Heneka, M.T., Golenbock, D.T., and Latz, E. (2015). Innate immunity in Alzheimer's disease. Nat Immunol 16, 229-236.
23. Heneka, M.T., Kummer, M.P., Stutz, A., Delekate, A., Schwartz, S., Vieira-Saecker, A., Griep, A., Axt, D., Remus, A., Tzeng, T.C., et al. (2013). NLRP3 is activated in Alzheimer's disease and contributes to pathology in APP/PS1 mice. Nature 493, 674-678.
24. Hornung, V., Bauernfeind, F., Halle, A., Samstad, E.O., Kono, H., Rock, K.L., Fitzgerald, K.A., and Latz, E. (2008). Silica crystals and aluminum salts activate the NALP3 inflammasome through phagosomal destabilization. Nat Immunol 9, 847-856.
25. Karch, J., and Molkentin, J.D. (2014). Identifying the components of the elusive mitochondrial permeability transition pore. Proc Natl Acad Sci USA 111, 10396-10397.
26. Karin, M., and Clevers, H. (2016). Reparative inflammation takes charge of tissue regeneration. Nature 529, 307-315.
27. Kim, J., Gupta, R., Blanco, L.P., Yang, S., Shteinfer-Kuzmine, A., Wang, K., Zhu, J., Yoon, H.E., Wang, X., Kerkhofs, M., et al. (2019). VDAC oligomers form mitochondrial pores to release mtDNA fragments and promote lupus-like disease. Science 366, 1531-1536.
28. Kotas, M.E., and Medzhitov, R. (2015). Homeostasis, inflammation, and disease susceptibility. Cell 160, 816-827.
29. Lam, N.Y., Rainer, T.H., Chiu, R.W., Joynt, G.M., and Lo, Y.M. (2004). Plasma mitochondrial DNA concentrations after trauma. Clin Chem 50, 213-216.
30. Lamkanfi, M., and Dixit, V.M. (2012). Inflammasomes and their roles in health and disease. Annu Rev Cell Dev Biol 28, 137-161.
31. Latz, E., Xiao, T.S., and Stutz, A. (2013). Activation and regulation of the inflammasomes. Nat Rev Immunol 13, 397-411.
32. Liu, S., Cai, X., Wu, J., Cong, Q., Chen, X., Li, T., Du, F., Ren, J., Wu, Y.T., Grishin, N.V., et al. (2015). Phosphorylation of innate immune adaptor proteins MAVS, STING, and TRIF induces IRF3 activation. Science 347, aaa2630.
33. Lu, A., Magupalli, V.G., Ruan, J., Yin, Q., Atianand, M.K., Vos, M.R., Schroder, G.F., Fitzgerald, K.A., Wu, H., and Egelman, E.H. (2014). Unified polymerization mechanism for the assembly of ASC-dependent inflammasomes. Cell 156, 1193-1206.
34. Mangan, M.S.J., Olhava, E.J., Roush, W.R., Seidel, H.M., Glick, G.D., and Latz, E. (2018). Targeting the NLRP3 inflammasome in inflammatory diseases. Nat Rev Drug Discov 17, 688.
35. Mao, G., Pan, X., Zhu, B.B., Zhang, Y., Yuan, F., Huang, J., Lovell, M.A., Lee, M.P., Markesbery, W.R., Li, G.M., et al. (2007). Identification and characterization of OGGI mutations in patients with Alzheimer's disease. Nucleic Acids Res 35, 2759-2766.
36. Matic, S., Jiang, M., Nicholls, T.J., Uhler, J.P., Dirksen-Schwanenland, C., Polosa, P.L., Simard, M.L., Li, X., Atanassov, I., Rackham, O., et al. (2018). Mice lacking the mitochondrial exonuclease MGME1 accumulate mtDNA deletions without developing progeria. Nat Commun 9, 1202.
37. McArthur, K., Whitehead, L.W., Heddleston, J.M., Li, L., Padman, B.S., Oorschot, V., Geoghegan, N.D., Chappaz, S., Davidson, S., San Chin, H., et al. (2018). BAK/BAX macropores facilitate mitochondrial herniation and mtDNA efflux during apoptosis. Science 359.
38. Murphy, M.P. (2018). Newly made mitochondrial DNA drives inflammation. Nature 560, 176-177.
39. Nakahira, K., Haspel, J.A., Rathinam, V.A., Lee, S.J., Dolinay, T., Lam, H.C., Englert, J.A., Rabinovitch, M., Cernadas, M., Kim, H.P., et al. (2011). Autophagy proteins regulate innate immune responses by inhibiting the release of mitochondrial DNA mediated by the NALP3 inflammasome. Nat Immunol 12, 222-230.
40. Oka, T., Hikoso, S., Yamaguchi, O., Taneike, M., Takeda, T., Tamai, T., Oyabu, J., Murakawa, T., Nakayama, H., Nishida, K., et al. (2012). Mitochondrial DNA that escapes from autophagy causes inflammation and heart failure. Nature 485, 251-255.
41. Parrish, J.Z., Yang, C., Shen, B., and Xue, D. (2003). CRN-1, a Caenorhabditis elegans FEN-1 homologue, cooperates with CPS-6/EndoG to promote apoptotic DNA degradation. EMBO J 22, 3451-3460.
42. Pastukh, V.M., Gorodnya, O.M., Gillespie, M.N., and Ruchko, M.V. (2016). Regulation of mitochondrial genome replication by hypoxia: The role of DNA oxidation in D-loop region. Free Radic Biol Med 96, 78-88.
43. Perez-Trevino, P., Velasquez, M., and Garcia, N. (2020). Mechanisms of mitochondrial DNA escape and its relationship with different metabolic diseases. Biochim Biophys Acta Mol Basis Dis 1866, 165761.
44. Ricci, C., Pastukh, V., Leonard, J., Turrens, J., Wilson, G., Schaffer, D., and Schaffer, S.W. (2008). Mitochondrial DNA damage triggers mitochondrial-superoxide generation and apoptosis. Am J Physiol Cell Physiol 294, C413-422.
45. Riley, J.S., Quarato, G., Cloix, C., Lopez, J., O'Prey, J., Pearson, M., Chapman, J., Sesaki, H., Carlin, L.M., Passos, J.F., et al. (2018). Mitochondrial inner membrane permeabilisation enables mtDNA release during apoptosis. EMBO J 37.
46. Riley, J.S., and Tait, S.W. (2020). Mitochondrial DNA in inflammation and immunity. EMBO Rep 21, e49799.
47. Rizzuto, R., Pinton, P., Carrington, W., Fay, F.S., Fogarty, K.E., Lifshitz, L.M., Tuft, R.A., and Pozzan, T. (1998). Close contacts with the endoplasmic reticulum as determinants of mitochondrial Ca2+ responses. Science 280, 1763-1766.
48. Sampath, H., Vartanian, V., Rollins, M.R., Sakumi, K., Nakabeppu, Y., and Lloyd, R.S. (2012). 8-Oxoguanine DNA glycosylase (OGG1) deficiency increases susceptibility to obesity and metabolic dysfunction. PLOS One 7, e51697.
49. Shimada, K., Crother, T.R., Karlin, J., Dagvadorj, J., Chiba, N., Chen, S., Ramanujan, V.K., Wolf, A.J., Vergnes, L., Ojcius, D.M., et al. (2012). Oxidized mitochondrial DNA activates the NLRP3 inflammasome during apoptosis. Immunity 36, 401-414.
50. Tanaka, Y., and Chen, Z.J. (2012). STING specifies IRF3 phosphorylation by TBK1 in the cytosolic DNA signaling pathway. Sci Signal 5, ra20.
51. Tumurkhuu, G., Chen, S., Montano, E.N., Ercan Laguna, D., De Los Santos, G., Yu, J.M., Lane, M., Yamashita, M., Markman, J.L., Blanco, L.P., et al. (2020). Oxidative DNA Damage Accelerates Skin Inflammation in Pristane-Induced Lupus Model. Front Immunol 11, 554725.
52. Tumurkhuu, G., Shimada, K., Dagvadorj, J., Crother, T.R., Zhang, W., Luthringer, D., Gottlieb, R.A., Chen, S., and Arditi, M. (2016). Ogg1-Dependent DNA Repair Regulates NLRP3 Inflammasome and Prevents Atherosclerosis. Circ Res 119, e76-90.
53. Vartanian, V., Tumova, J., Dobrzyn, P., Dobrzyn, A., Nakabeppu, Y., Lloyd, R.S., and Sampath, H. (2017). 8-oxoguanine DNA glycosylase (OGG1) deficiency elicits coordinated changes in lipid and mitochondrial metabolism in muscle. PLOS One 12, e0181687.
54. von Kleist, L., Stahlschmidt, W., Bulut, H., Gromova, K., Puchkov, D., Robertson, M.J., MacGregor, K.A., Tomilin, N., Pechstein, A., Chau, N., et al. (2011). Role of the clathrin terminal domain in regulating coated pit dynamics revealed by small molecule inhibition. Cell 146, 471-484.
55. Wang, W., Esbensen, Y., Kunke, D., Suganthan, R., Rachek, L., Bjoras, M., and Eide, L. (2011). Mitochondrial DNA damage level determines neural stem cell differentiation fate. J Neurosci 31, 9746-9751.
56. Wang, Y., Ning, X., Gao, P., Wu, S., Sha, M., Lv, M., Zhou, X., Gao, J., Fang, R., Meng, G., et al. (2017). Inflammasome Activation Triggers Caspase-1-Mediated Cleavage of cGAS to Regulate Responses to DNA Virus Infection. Immunity 46, 393-404.
57. West, A.P., Khoury-Hanold, W., Staron, M., Tal, M.C., Pineda, C.M., Lang, S.M., Bestwick, M., Duguay, B.A., Raimundo, N., MacDuff, D.A., et al. (2015). Mitochondrial DNA stress primes the antiviral innate immune response. Nature 520, 553-557.
58. West, A.P., and Shadel, G.S. (2017). Mitochondrial DNA in innate immune responses and inflammatory pathology. Nat Rev Immunol 17, 363-375.
59. White, M.J., McArthur, K., Metcalf, D., Lane, R.M., Cambier, J.C., Herold, M.J., van Delft, M.F., Bedoui, S., Lessene, G., Ritchie, M.E., et al. (2014). Apoptotic caspases suppress mtDNA-induced STING-mediated type I IFN production. Cell 159, 1549-1562.
60. Wu, Z., Sainz, A.G., and Shadel, G.S. (2021). Mitochondrial DNA: cellular genotoxic stress sentinel. Trends Biochem Sci 46, 812-821.
61. Xian, H., and Liou, Y.C. (2019). Loss of MIEF1/MiD51 confers susceptibility to BAX-mediated cell death and PINK1-PRKN-dependent mitophagy. Autophagy 15, 2107-2125.
62. Xian, H., Liu, Y., Rundberg Nilsson, A., Gatchalian, R., Crother, T.R., Tourtellotte, W.G., Zhang, Y., Aleman-Muench, G.R., Lewis, G., Chen, W., et al. (2021). Metformin inhibition of mitochondrial ATP and DNA synthesis abrogates NLRP3 inflammasome activation and pulmonary inflammation. Immunity 54, 1463-1477 e1411.
63. Xian, H., Yang, Q., Xiao, L., Shen, H.M., and Liou, Y.C. (2019). STX17 dynamically regulated by Fis1 induces mitophagy via hierarchical macroautophagic mechanism. Nat Commun 10, 2059.
64. Xiao, L., Xian, H., Lee, K.Y., Xiao, B., Wang, H., Yu, F., Shen, H.M., and Liou, Y.C. (2015). Death-associated Protein 3 Regulates Mitochondrial-encoded Protein Synthesis and Mitochondrial Dynamics. J Biol Chem 290, 24961-24974.
65. Yu, C.H., Davidson, S., Harapas, C.R., Hilton, J.B., Mlodzianoski, M.J., Laohamonthonkul, P., Louis, C., Low, R.R.J., Moecking, J., De Nardo, D., et al. (2020). TDP-43 Triggers Mitochondrial DNA Release via mPTP to Activate cGAS/STING in ALS. Cell 183, 636-649 e618.
66. Yu, Y., Liu, Y., An, W., Song, J., Zhang, Y., and Zhao, X. (2019). STING-mediated inflammation in Kupffer cells contributes to progression of nonalcoholic steatohepatitis. J Clin Invest 129, 546-555.
67. Zhang, Q., Raoof, M., Chen, Y., Sumi, Y., Sursal, T., Junger, W., Brohi, K., Itagaki, K., and Hauser, C.J. (2010). Circulating mitochondrial DAMPs cause inflammatory responses to injury. Nature 464, 104-107.
68. Zheng, L., Jia, J., Finger, L.D., Guo, Z., Zer, C., and Shen, B. (2011). Functional regulation of FEN1 nuclease and its link to cancer. Nucleic Acids Res 39, 781-794.
69. Zhong, F., Liang, S., and Zhong, Z. (2019). Emerging Role of Mitochondrial DNA as a Major Driver of Inflammation and Disease Progression. Trends Immunol 40, 1120-1133.
70. Zhong, S., Ng, M.C., Lo, Y.M., Chan, J.C., and Johnson, P.J. (2000). Presence of mitochondrial tRNA (Leu (UUR)) A to G 3243 mutation in DNA extracted from serum and plasma of patients with type 2 diabetes mellitus. J Clin Pathol 53, 466-469.
71. Zhong, Z., Liang, S., Sanchez-Lopez, E., He, F., Shalapour, S., Lin, X.J., Wong, J., Ding, S., Seki, E., Schnabl, B., et al. (2018). New mitochondrial DNA synthesis enables NLRP3 inflammasome activation. Nature 560, 198-203.
72. Zhong, Z., Umemura, A., Sanchez-Lopez, E., Liang, S., Shalapour, S., Wong, J., He, F., Boassa, D., Perkins, G., Ali, S.R., et al. (2016). NF-kappaB Restricts Inflammasome Activation via Elimination of Damaged Mitochondria. Cell 164, 896-910.
73. Zhou, R., Yazdi, A.S., Menu, P., and Tschopp, J. (2011). A role for mitochondria in NLRP3inflammasome activation. Nature 469, 221-225.

What is claimed is:

1. A method for one or more in a mammalian cell: inhibition of mtDNA fragmentation;
inhibition of cytostolic Ox-mtDNA; inhibition of mtDNA release; or inhibition of Casp1 activation and IL-1β production, comprising contacting the mammalian cell with an effective amount of an inhibitor of human flap endonuclease-1 (FEN1), thereby inhibiting mtDNA fragmentation; inhibiting cytostolic Ox-mtDNA; inhibiting mtDNA release; or inhibiting of Casp1 activation and IL-1β production.

2. The method of claim 1, wherein the inhibitor of human flap endonuclease-1 (FEN1) is a small molecule or an inhibitory oligonucleotide.

3. The method of claim 1, wherein the mammalian cells is selected from a human cell, a murine cell, a rat cell, a canine cell, a feline cell, or a bovine cell.

4. The method of claim 1, wherein the contacting is in vivo or in vitro.

5. The method of claim 1, wherein the inhibitor of FEN1 is FEN-IN-4.

6. The method of claim 1, wherein the inhibitor of FEN1 further comprises a carrier or a pharmaceutically acceptable carrier.

7. A method for treating a disease or condition associated with mtDNA fragmentation; cytostolic Ox-mtDNA; mtDNA release; or Casp1 activation and IL-1β production in a subject in need thereof, comprising administering to the subject an effective amount of an inhibitor of human flap endonuclease-1 (FEN1), thereby treating the disease or condition associated with mtDNA fragmentation; cytostolic Ox-mtDNA; mtDNA release; or Casp1 activation and IL-1β production in the subject.

8. The method of claim 7, wherein the disease or condition is an inflammatory or a neurodegenerative disease.

9. The method of claim 8, wherein the disease or condition is selected from chronic diseases and metabolic pathologies, optionally obesity, type 2 diabetes, atherosclerosis, inflammatory bowel diseases, gout, and periodic fevers, as well as neurodegenerative disorders, including Parkinson's and Alzheimer's diseases (AD), atherosclerosis, NASH, asthma, inflammatory bowel disease, or melanoma.

10. The method of claim 7, wherein the inhibitor of human flap endonuclease-1 (FEN1) is a small molecule or an inhibitory oligonucleotide.

11. The method of claim 7, wherein the subject is a mammal.

12. The method of claim 11, wherein the mammal is selected from a human patient, a murine, a rat, a canine, a feline, or a bovine.

13. The method of claim 7, wherein the inhibitor of FEN1 is FEN-IN-4.

14. The method of claim 7, wherein the inhibitor of FEN1 further comprises a carrier or a pharmaceutically acceptable carrier.


SEQUENCE LISTING (41 sequences)

SEQ ID NO: 1 - DNA, 23 bp, synthetic construct
  source 1..23: mol_type = other DNA
aatctaccatcctccgtgaaacc

SEQ ID NO: 2 - DNA, 25 bp, synthetic construct
  source 1..25: mol_type = other DNA
tcagtttagctacccccaagtttaa

SEQ ID NO: 3 - DNA, 20 bp, synthetic construct
  source 1..20: mol_type = other DNA
tagagggacaagtggcgttc

SEQ ID NO: 4 - DNA, 18 bp, synthetic construct
  source 1..18: mol_type = other DNA
cgctgagccagtcagtgt

SEQ ID NO: 5 - DNA, 25 bp, synthetic construct
  source 1..25: mol_type = other DNA
ctagctcatgtgtcaagaccctctt

SEQ ID NO: 6 - DNA, 20 bp, synthetic construct
  source 1..20: mol_type = other DNA
gccagcacgtttctctcgtt

SEQ ID NO: 7 - DNA, 20 bp, synthetic construct
  source 1..20: mol_type = other DNA
gccccagatatagcattccc

SEQ ID NO: 8 - DNA, 20 bp, synthetic construct
  source 1..20: mol_type = other DNA
gttcatcctgttcctgctcc

SEQ ID NO: 9 - DNA, 20 bp, synthetic construct
  source 1..20: mol_type = other DNA
ctagaaaccccgaaaccaaa

SEQ ID NO: 10 - DNA, 20 bp, synthetic construct
  source 1..20: mol_type = other DNA
ccagctatcaccaagctcgt

SEQ ID NO: 11 - DNA, 20 bp, synthetic construct
  source 1..20: mol_type = other DNA
atgggaagccgaacatactg

SEQ ID NO: 12 - DNA, 20 bp, synthetic construct
  source 1..20: mol_type = other DNA
cagtctcagtgggggtgaat

SEQ ID NO: 13 - DNA, 20 bp, synthetic construct
  source 1..20: mol_type = other DNA
ctggtgaaaaggacctctcg

SEQ ID NO: 14 - DNA, 26 bp, synthetic construct
  source 1..26: mol_type = other DNA
tgaagtactcattatagtcaagggca

SEQ ID NO: 15 - DNA, 19 bp, synthetic construct
  source 1..19: mol_type = other DNA
agttgacggaccccaaaag

SEQ ID NO: 16 - DNA, 20 bp, synthetic construct
  source 1..20: mol_type = other DNA
agctggatgctctcatcagg

SEQ ID NO: 17 - DNA, 21 bp, synthetic construct
  source 1..21: mol_type = other DNA
ccaggtagctatggtactcca

SEQ ID NO: 18 - DNA, 21 bp, synthetic construct
  source 1..21: mol_type = other DNA
gctaccaaactggctataatc

SEQ ID NO: 19 - DNA, 21 bp, synthetic construct
  source 1..21: mol_type = other DNA
ccctcacactcagatcatctt

SEQ ID NO: 20 - DNA, 19 bp, synthetic construct
  source 1..19: mol_type = other DNA
gctacgacgtgggctacag

SEQ ID NO: 21 - DNA, 22 bp, synthetic construct
  source 1..22: mol_type = other DNA
atcatccctgcgagcctatcct

SEQ ID NO: 22 - DNA, 24 bp, synthetic construct
  source 1..24: mol_type = other DNA
gaccttttttggctaaacgctttc

SEQ ID NO: 23 - DNA, 22 bp, synthetic construct
  source 1..22: mol_type = other DNA
catcctggtgaggaacgaaagg

SEQ ID NO: 24 - DNA, 22 bp, synthetic construct
  source 1..22: mol_type = other DNA
ctcagccagaactggtcttcgt

SEQ ID NO: 25 - DNA, 22 bp, synthetic construct
  source 1..22: mol_type = other DNA
gctcaggcttacgttgacaagg

SEQ ID NO: 26 - DNA, 22 bp, synthetic construct
  source 1..22: mol_type = other DNA
ctttaggcgtgtccatccttcc

SEQ ID NO: 27 - DNA, 22 bp, synthetic construct
  source 1..22: mol_type = other DNA
cctctgctttctagtgatgccg

SEQ ID NO: 28 - DNA, 22 bp, synthetic construct
  source 1..22: mol_type = other DNA
cgtaaacacggtcttgctcctg

SEQ ID NO: 29 - DNA, 25 bp, synthetic construct
  source 1..25: mol_type = other DNA
gtgttatctgacatacaccatacag

SEQ ID NO: 30 - DNA, 25 bp, synthetic construct
  source 1..25: mol_type = other DNA
tgggaactactagaattgatcagga

SEQ ID NO: 31 - DNA, 23 bp, synthetic construct
  source 1..23: mol_type = other DNA
atgatttatctcaaccctagcag

SEQ ID NO: 32 - DNA, 26 bp, synthetic construct
  source 1..26: mol_type = other DNA
gtagtgggacttctagagggttaagt

SEQ ID NO: 33 - DNA, 28 bp, synthetic construct
  source 1..28: mol_type = other DNA
actgaaaatgcttagatggataattgta

SEQ ID NO: 34 - DNA, 29 bp, synthetic construct
  source 1..29: mol_type = other DNA
gtatgattagagttttggttcacggaaca

SEQ ID NO: 35 - DNA, 58 bp, synthetic construct
  source 1..58: mol_type = other DNA
ccggcaagtcagaaagacttaacatctcgagatgttaagtctttctgacttgtttttg

SEQ ID NO: 36 - DNA, 58 bp, synthetic construct
  source 1..58: mol_type = other DNA
ccgggatgtccatgtatggcagattctcgagaatctgccatacatggacatctttttg

SEQ ID NO: 37 - DNA, 59 bp, synthetic construct
  source 1..59: mol_type = other DNA
ccgggcccagtctttgaattgagatctcgagatctcaattcaaagactgggcttttttg

SEQ ID NO: 38 - DNA, 58 bp, synthetic construct
  source 1..58: mol_type = other DNA
ccggcgccatattgatgctctggaactcgagttccagagcatcaatatggcgtttttg

SEQ ID NO: 39 - DNA, 58 bp, synthetic construct
  source 1..58: mol_type = other DNA
ccgggaatgacatcaagagctacttctcgagaagtagctcttgatgtcattctttttg

SEQ ID NO: 40 - DNA, 58 bp, synthetic construct
  source 1..58: mol_type = other DNA
ccgggagttgataaataccacgttactcgagtaacgtggtatttatcaactctttttg

SEQ ID NO: 41 - DNA, 58 bp, synthetic construct
  source 1..58: mol_type = other DNA
ccggtgactcttgataccatatttgctcgagcaaatatggtatcaagagtcatttttg